(19) United States
(12) Patent Application Publication
(10) Pub. No.: US 2022/0323017 A1
(43) Pub. Date: Oct. 13, 2022

(54) PROCESS AND DEVICE FOR THE APPROXIMATE DETERMINATION OF HEARTBEAT TIMES

(71) Applicant: Drägerwerk AG & Co. KGaA, Lübeck (DE)

(72) Inventors: Marcus EGER, Lübeck (DE); Lorenz KAHL, Lübeck (DE)

(21) Appl. No.: 17/707,476

(22) Filed: Mar. 29, 2022

(30) Foreign Application Priority Data
Mar. 30, 2021 (DE) 10 2021 107 948.9

(51) Int. Cl. A61B 5/00; A61B 5/0205; A61B 5/024; A61B 5/08; A61M 16/00

(52) U.S. Cl. CPC A61B 5/7221; A61B 5/0205; A61B 5/024; A61B 5/0816; A61M 16/024; A61M 2230/42; A61M 2230/06

(57) ABSTRACT

A process and a signal processing unit (5) approximately detect a respective characteristic heartbeat time {H_Zp[f](x), H_Zp[s](x₁), . . . , H_Zp[s](x_{N})} per heartbeat for a sequence of heartbeats of a patient (P). A sensor array (2.1, 2.2) sends at least one sum signal [Sig_{Sum}(1), Sig_{Sum}(2)], which results from a superimposition of a cardiogenic signal and of a respiratory signal. A first detector (25.1) calculates a respective first detection result for each characteristic heartbeat time, and a second detector (25.2, . . . ) calculates a second detection result. The first detector (25.1) analyzes a different sum signal and/or applies a different method of analysis than the second detector (25.2, . . . ). The signal processing unit (5) calculates a respective estimation (representation) for each heartbeat time and uses this estimation as the characteristic heartbeat time. The signal processing unit (5) uses a first detection result and a second detection result to calculate the estimation.

### CROSS REFERENCE TO RELATED APPLICATIONS

This application claims the benefit of priority under 35 U.S.C. § 119 of German Application 10 2021 107 948.9, filed Mar. 30, 2021, the entire contents of which are incorporated herein by reference.

### TECHNICAL FIELD

The present invention pertains to a process and to a signal processing unit, which are configured to automatically approximately determine, in particular represent a respective characteristic heartbeat time per heartbeat for a sequence of heartbeats of a patient.

### TECHNICAL BACKGROUND

The present invention can be applied, for example, for mechanical/artificial ventilation (in short: “ventilation”) of a patient. A fluid connection is established between the patient and a ventilator. The ventilator performs a sequence of ventilation strokes and feeds a gas mixture to the patient during each ventilation stroke. This gas mixture that flows through the fluid connection, comprises oxygen and may additionally contain at least one anesthetic. If the patient is not fully sedated, the patient's diaphragm muscles perform an intrinsic breathing activity. In case the intrinsic breathing activity is sufficiently intense, the ventilation shall, as a rule, be synchronized with the intrinsic breathing activity of the patient. The ventilation shall thus be assisted ventilation. In many cases, a respiratory signal, i.e., a signal which correlates with the intrinsic breathing activity of the patient, i.e., with the breathing activity which is brought about by the diaphragm muscles of the patient, is needed for this.

The respiratory signal cannot, as a rule, be measured directly. It is possible, by contrast, to measure a sum signal, for example, with measuring electrodes on the skin of the patient or with a probe or catheter in the trachea or in the stomach. This sum signal results from a superimposition of the respiratory signal being sought with a cardiogenic signal. The cardiogenic signal is caused by the cardiac activity of the patient. In order to calculate the respiratory signal by using the sum signal, it is necessary to eliminate/subtract the cardiogenic signal from the sum signal, i.e., to compensate the contribution of the cardiogenic signal to the sum signal by calculation. A respective characteristic heartbeat time is needed for each heartbeat for this purpose.

Another application of the present invention is that of detecting the heart rate variability of the cardiac activity of the patient. The interval between two consecutive heartbeat times even varies during regular cardiac activity, as a rule. Both a too low and a too high variability can be detected automatically by means of the present invention.

A process and a device, which are capable of measuring and monitoring the cardiac activity of a patient, are described in US 2019/0231273 A 1. A first sensor is capable of measuring a physiological signal at a first measuring point on the body of the patient, and a second sensor is capable of measuring a physiological signal at a second measuring point. A heart sound signal and a respiration signal are extracted from the measured values (sensed data 236) of the sensors. The probability that a physiological event will occur is determined in one embodiment. The occurrence of the next heartbeat, the occurrence of cardiac failure or the loss of consciousness are mentioned as examples of such events, cf. Par. [0108].

### SUMMARY

A basic object of the present invention is to provide a process as well as a signal processing unit for automatically approximately determining, in particular representing a respective characteristic heartbeat time for a sequence of heartbeats of a patient, which deliver estimations (representations) for the heartbeat times with greater reliability than prior-art processes and signal processing units.

The present invention is accomplished by a process with the features described herein and by a signal processing unit with the features described herein. Advantageous embodiments are described herein. Advantageous embodiments of the process according to the present invention are, if meaningful, advantageous embodiments of the signal processing unit according to the present invention, of the arrangement according to the present invention and of the system according to the present invention, and vice versa, advantageous embodiments of the signal processing unit are also advantageous embodiments of the process.

According to the present invention, the signal processing unit approximately detects a respective characteristic heartbeat time for a sequence of heartbeats of a patient. The term “approximately detect a characteristic heartbeat time” designates the process of detecting the heartbeat and of at least approximately determining the characteristic heartbeat time. A cardiogenic signal, i.e., a signal, which correlates with the cardiac activity of the patient, has a typical temporal course (curve) in the course of a single heartbeat. This course is repeated, optionally with slight variations, for each heartbeat. A characteristic point of this typical course, for example, the R peak or the middle of the QRS phase, is used as the characteristic heartbeat time of this heartbeat. The signal processing unit detects this characteristic heartbeat time after the heart muscles of the patient have fully carried out or at least begun this heartbeat. A heartbeat has begun, for example, when the P peak is reached, and it is ended when the T wave has subsided. The signal processing unit thus detects a time of an event, the beginning of which is in the past. It is possible that the approximate detection for the characteristic heartbeat time is already present before the heartbeat has been fully concluded. It is also possible that the approximate detection is present not earlier than after conclusion of the heartbeat.

The data-processing signal processing unit according to the present invention comprises at least one first detector and at least one second detector. The process according to the present invention is carried out automatically using such a signal processing unit.

The process is carried out, furthermore, using a sensor arrangement comprising at least one sensor array. The sensor array or each sensor array comprises at least one respective sensor, preferably an electrically operating sensor, and measures a respective variable, which variable correlates with the cardiac activity and/or with the breathing activity of the patient. The measurement or each measurement of the variable or of a correlating variable delivers measured values. At least one sum signal is generated by using measured values of the sensor array or of at least one, preferably of each sensor array, wherein a respective measured value processing is preferably carried out per sensor array. The processing of measured values or each processing of measured values preferably comprises the step that a raw signal as well as an average course or a course as a function of isoelectric points are calculated from the measured values, and the average course or the course, which depends on isoelectric points, is subtracted from the raw signal (baseline filtering).

The sum signal or each sum signal comprises a superimposition of a cardiogenic signal and of a respiratory signal and is optionally influenced by at least one additional signal (unwanted signal, interference signal). The cardiogenic signal correlates with the cardiac activity of the patient. The respiratory signal correlates with the intrinsic breathing activity of the patient (the patient's own breathing activity). The intrinsic breathing activity of the patient is generated by the intrinsic respiratory muscles of the patient and comprises the spontaneous breathing of the patient, which is triggered by electrical pulses generated in the body of the patient, as well as optionally a stimulated breathing activity, which is likewise carried out by the intrinsic respiratory muscles, but is stimulated externally, for example, in a magnetic field. Ha plurality of sum signals are used, then each sum signal comprises a respective superimposition of a cardiogenic signal and of a respiratory signal, wherein an additional signal (unwanted signal) optionally acts on each sum signal and wherein the unwanted signals may be different from sum signal to sum signal. The cardiogenic signal or each cardiogenic signal correlates with the same cardiac activity, and the respiratory signal or each respiratory signal correlates with the same intrinsic breathing activity.

The signal processing unit according to the present invention is configured to receive measured values from the sensor array or from each sensor array and to generate a sum signal from the measured values received or at least from some of the received measured values, wherein the signal processing unit preferably processes received measured values.

The process according to the present invention comprises the following steps, which are carried out automatically, and the signal processing unit according to the present invention is configured to carry out the following steps automatically:

The first detector or each first detector calculates a respective first detection result for each characteristic heartbeat time, wherein the first detection result is a first estimation for the heartbeat time. It is possible that the first detector or a first detector does not detect individual heartbeat times, i.e., “overlooks” individual heartbeats. The first detector or each first detector uses a sum signal to calculate the first detection results.

The second detector or each second detector calculates a respective second detection result for each characteristic heartbeat time, wherein the second detection result is a second estimation (representation) for the same characteristic heartbeat time. Both estimations (representations) thus pertain to the same heartbeat. It is possible that the second detector or a second detector does not detect individual characteristic heartbeat times. The second detector or each second detector uses a respective sum signal to calculate the second detection results. The second detector may use the same sum signal as the first detector or a different sum signal than the first detector. If two sum signals are used, then these originate from the same patient and pertain to the same time period or at least to overlapping time periods.

In an alternative, the first detector or each first detector analyzes a different sum signal than the second detector or each second detector. These two sum signals are generated by using measured values from different sensor arrays for the same patient in a first embodiment. The two sum signals are generated by different methods of processing measured values being applied to the measured values from the same sensor array in a second embodiment. These two embodiments can be combined with one another.

For example, a plurality of sensor arrays measure measured values at different measuring points in the body or at the body of the patient, and a respective sum signal is generated from the measured values of each sensor array. It is possible that a plurality of first detectors analyze the same first sum signal, and/or a plurality of second detectors analyze the same second sum signal, but with different methods of detection. It is also possible that different first detectors and/or second detectors analyze different sum signals.

In another alternative, the first detector or each first detector applies a different method of analysis to a sum signal than the second detector or each second detector. It is possible that all detectors or at least one first detector and one second detector analyze the same sum signal, but then with different methods of analysis.

The two alternatives, namely using two different sum signals and applying two different methods of analysis to a sum signal, can be combined. In particular, it is possible that a plurality of first detectors analyze the same first sum signal with different methods of analysis. A different combination possibility is that a plurality of second detectors analyze the same second sum signal or different second sum signals with different methods of analysis.

It is also possible that more than two sum signals are analyzed, especially either with the same method of analysis or with at least two different methods of analysis. For example, the methods of analysis need different periods to deliver a result.

The signal processing unit automatically calculates at least one respective estimation for each heartbeat and thus for each characteristic heartbeat time. The estimation or each estimation approximately indicates the characteristic heartbeat time. The estimation or an estimation or an aggregation of a plurality of estimations is used as the characteristic heartbeat time.

In one embodiment, the calculated characteristic heartbeat time or each calculated characteristic heartbeat time is in the past. More precisely: The calculation result is chronologically present after the characteristic heartbeat time, even after the end of the heartbeat in one embodiment. In another embodiment, the calculation result is present after the heartbeat was begun and before it was concluded, and especially optionally before the characteristic heartbeat time. The term “characteristic heartbeat time” identifies the actual characteristic time of the heartbeat. The identifications “approximate” and “estimation” specify that the characteristic heartbeat time cannot, as a rule, be detected precisely and is estimated or represented. The time interval between the beginning of a heartbeat, for example, the P peak or the beginning of the P wave, as well as the characteristic heartbeat time remains, as a rule, largely constant from heartbeat to heartbeat, so that it is in many cases possible to provide the calculation result already before the end of the heartbeat or even before the characteristic heartbeat time.

An estimation for the actual characteristic heartbeat time can be used, for example, for the following applications:
A respiratory signal, which correlates with the intrinsic breathing activity of the patient, is being sought. For example, the respiratory signal is needed to synchronize the ventilation strokes of a ventilator with the intrinsic breathing activity of the patient or to monitor the intrinsic breathing activity of the patient. For example, a neuro-muscular efficiency of the respiratory muscles of the patient shall be determined, i.e., how well the respiratory muscles convert electrical signals, which are generated in the body of the patient, into pneumatic breathing activity.
As a rule, the respiratory signal cannot be measured directly. Rather, at least one sum signal is generated, preferably by the sensor array or a sensor array according to the present invention. This sum signal results from a superimposition of the respiratory signal being sought with a cardiogenic signal as well as optionally with at least one unwanted signal or with another disturbance variable. To obtain the respiratory signal, the contribution of the cardiogenic signal to the sum signal is compensated by calculation, i.e. is eliminated. For this, the actual characteristic heartbeat times are needed. For example, a respective cardiogenic reference signal segment, which is positioned in a correctly timed manner, is subtracted from the sum signal per heartbeat to obtain the respiratory signal. Such a method has become known by the designation “template subtraction.”
The cardiac activity of the patient shall be monitored, for which monitoring the cardiogenic signal is needed. In particular, both irregularities and heart failure shall be detected.

How precisely the heartbeat time shall be determined depends on the respective application. One already mentioned application is the application that the respiratory signal shall be determined and the effect of the cardiogenic signal on the sum signal shall be compensated by calculation for this. An accuracy of less than 10 msec is needed for this in many cases.

It would be conceivable to predict a heartbeat, i.e., to provide the characteristic heartbeat time, before this heartbeat has begun. As a rule, the cardiac activity of a patient is an approximately periodic process, but usually not a precisely periodic process. Therefore, a characteristic heartbeat time often cannot be determined solely by a prediction with sufficient reliability and accuracy, for example, as follows:
The heartbeat is understood to be a periodic process or to be a superimposition of a plurality of periodic processes.
The frequency and optionally additional parameters of this periodic process are calculated for the periodic process or for each periodic process.

“Reliability” is defined as each heartbeat actually being detected and no false detection occurring. “Accuracy” means that the determined characteristic heartbeat time coincides sufficiently accurately with the actual characteristic heartbeat time, in many applications with an accuracy below I msec, often even below 0.1 msec.

Namely, irregularities of the cardiac activity could often not be detected with sufficient accuracy to predict a heartbeat time by means of such a process. In addition, such a process requires that a model for the cardiac activity be assumed to be true. Other possible processes for predicting a characteristic heartbeat time require that the distribution of the heartbeat times follow a predefined probability distribution, for example, a normal distribution. Such a process also requires that a model be assumed to be true, without testing this assumption.

The process according to the present invention and the signal processing unit according to the present invention detect, by contrast, in many cases each characteristic heartbeat time that has already taken place with sufficient reliability and accuracy and sufficiently quickly. In many cases the present invention does not require that the actual cardiac activity be precisely a periodic process or a superimposition of a plurality of periodic processes. This is possible because the present invention does not deliver a characteristic heartbeat time for a heartbeat that has not yet begun. However, at least one detector may use such a model assumption.

The process according to the present invention and the signal processing unit detect a respective characteristic heartbeat time for a sequence of heartbeats. They use at least one sum signal for this. The sum signal or each sum signal comprises or is a superimposition of a cardiogenic signal and of the respiratory signal.

In one embodiment of the present invention, the cardiogenic signal is determined. As was already mentioned, this cardiogenic signal can especially be used for monitoring the cardiac activity of the patient or for compensating by calculation (eliminating) the contribution of the cardiogenic signal to the sum signal.

In a first alternative to this embodiment, a cardiogenic signal segment is predefined and preferably stored in a memory, to which the signal processing unit has at least temporarily reading access. In a second alternative a cardiogenic signal segment is determined by using a sample. The predefined or determined cardiogenic signal segment approximately describes the temporal course of the cardiac activity of the patient in the course of a single heartbeat. The sample comprises a plurality of segments of the sum signal or of a sum signal, wherein each segment pertains to a respective heartbeat time period and this heartbeat time period is in the past. The sample preferably comprises at least 40 segments. Because the sum signal is essentially determined by the cardiogenic signal during the heartbeat time period and the effect of the respiratory signal and of each possible unwanted signal is relatively low, this sum signal segment largely coincides with the course of the cardiogenic signal in the heartbeat time period.

In one preferred embodiment, the determined cardiogenic signal segment is updated continuously. For this purpose, the respective last (previous) N sum signal segments are used to calculate a cardiogenic signal segment again. This embodiment takes into consideration possible changes in the cardiac activity and/or in the breathing activity of the patient.

The signal processing unit determines the cardiogenic signal. For this, it uses, on the one hand, the predefined or determined cardiogenic signal segment and, on the other hand, the heartbeat times determined according to the present invention. The cardiogenic signal segments are positioned in a correctly timed manner, for which the determined characteristic heartbeat times are used and combined to form the cardiogenic signal. A gap between two consecutive cardiogenic signal segments is filled by suitable interpolation as needed.

In a variant of this embodiment, the signal processing unit determines the respiratory signal. For this, it subtracts the cardiogenic signal, which was determined as just described, from the sum signal or from a sum signal or even from a combination of a plurality of sum signals. Possible applications of the cardiogenic signal were described above.

It is possible that the signal processing unit calculates a plurality of estimations (representations) for the same characteristic heartbeat time, for example, by analyzing different sum signals, with different calculation methods and/or in calculation periods of different duration. Optionally, a plurality of estimations arc combined into one estimation for the same characteristic heartbeat time. For example, an arithmetic mean or a weighted mean or a median of a plurality of estimations or the estimation with the greatest reliability is used as a characteristic heartbeat time. The weighting factors depend on the respective reliability of an estimation. The reliability describes the degree of accordance between the actual heartbeat time and the respective characteristic heartbeat time which a detector has calculated. This degree of accordance can be determined only in retrospect. of course. It is possible that different estimations of the same characteristic heartbeat time are used for different purposes.

For each estimation, the signal processing unit uses at least one first detection result, i.e., a detection result from the first detector or from a first detector, as well as at least one second detection result, i.e., a detection result from the second detector or from a second detector.

According to the present invention, a first detector and a second detector are used. Each detector detects the characteristic heartbeat times in the sum signal or in a respective sum signal, and more precisely, it determines approximately the characteristic heartbeat times. The estimations of the at least two detectors for the characteristic heartbeat times may also be different for one and the same heartbeat.

In an alternative, the first detector or each first detector analyzes a respective first sum signal. The second detector or each second detector analyzes a respective second sum signal which is different from the first sum signal. Each first detector thus analyzes a different sum signal than each second detector. The different sum signals are obtained, for example, by differently positioned sensors or by sensors, which apply different measuring methods, or by different processing of the same measured values. It is possible that the same detector is applied, preferably alternately, to a first sum signal (as the first detector) and then to a second sum signal (as the second detector) one after the other. In this case as well, a first detector and a second detector are mentioned.

The first detector or each first detector applies a different method of analysis than the second detector or each second detector in another alternative. Each first detector thus applies a different method than each second detector for approximately detecting characteristic heartbeat times. It is possible that a first detector and a second detector analyze the same sum signal, but then with different methods of analysis.

Both embodiments lead in many cases to a higher reliability than a process, in which a single detector analyzes a signal sum signal.

In one preferred embodiment, measured values from a sensor arrangement comprising two different sensor arrays with differently positioned sensors are used. Each sensor of the first sensor array is located in a different position than each sensor of the second sensor array, and especially in a different position in relation to the causing signal sources in the body of the patient, especially in relation to the heart muscles and/or to the respiratory muscles. In an alternative to this preferred embodiment, a sensor arrangement comprises the same sensor array that delivers measured values, from which the signal processing unit generates two different sum signals, wherein the sensor arrangement applies different measuring methods and/or different methods for signal processing to deliver measured values for the different sum signals.

At least two different sum signals, which pertain to the same patient and to the same time period and both comprise a superimposition of a respiratory signal and a cardiogenic signal, are available in both cases of the embodiment with a plurality of sensor arrays. However, the signal sources coincide because the sum signals are generated from the same heart muscles and from the same respiratory muscles of the same patient. Since at least two sum signals from at least two sensor arrays, which are positioned differently and/or operate differently, are used, the reliability increases compared with an embodiment in which only a single sum signal is used. Thanks to the present invention and thanks to the embodiment with different sensor arrays the effect of unwanted (interference) signals on the determination of the characteristic heartbeat time is in many cases compensated by calculation up to a certain degree.

According to the present invention, the signal processing unit comprises at least one first detector and at least one second detector. In one embodiment, the signal processing unit comprises a first real time detector, an additional first detector, a second real time detector and an additional second detector. Overall, the signal processing unit thus comprises two first detectors and two second detectors. The signal processing unit comprises two real time detectors and two additional detectors.

According to this embodiment, a calculation period is predefined. Preferably, this calculation period is predefined such that no more than this calculation period may occur between a characteristic heartbeat time and the provision (by calculation) of the estimations for this characteristic heartbeat time. In the calculation period, the first real time detector delivers a respective first real time detection result for each characteristic heartbeat time, and the second real time detector delivers a respective second real time detection result. The two real time detectors preferably operate parallel to one another. The signal processing unit calculates in the calculation period a respective real time estimation for the characteristic heartbeat time of each heartbeat. For this real time estimation (representation), the signal processing unit uses the first real time detection result and the second real time detection result. In one embodiment, the calculation period is shorter than the heartbeat time period. Hence, two calculations of the two real detectors are available before the heartbeat has concluded.

In addition, the additional first detector calculates an additional first detection result, and the additional second detector calculates an additional second detection result. The signal processing unit calculates an additional estimation for the respective characteristic heartbeat time of each heartbeat. For this, the signal processing unit uses the additional first detection result and the additional second detection result. The additional estimation is not necessarily already available in the calculation period. Since more computing time and in many cases a longer segment of the sum signal or of a sum signal are available, the additional estimation has a higher reliability than the real time estimation.

On the one hand, this embodiment makes it possible to calculate an estimation for the characteristic heartbeat time in the predefined calculation period. This real time estimation can be used to meet predefined real time requirements, for example, to calculate a respiratory signal in the calculation period and to actuate a ventilator using this respiratory signal, so that the ventilator carries out a sequence of ventilation strokes (ventilation phases) and these ventilation strokes are well synchronized with the intrinsic breathing activity of the patient. Performing synchronized ventilation requires that the respiratory signal be present in a calculation period, so that the ventilation strokes, which are triggered by the intrinsic breathing activity of the patient, lag behind the breaths of the patient only by this calculation period.

Preferably the calculation period is determined to be so short that the real time estimation is present for a characteristic heartbeat time before this heartbeat is completed. This embodiment makes it easier to synchronize the ventilation strokes of a ventilator with the intrinsic breathing activity. The additional estimation is in many cases present only after the calculation period, i.e., as a rule, only if the heartbeat is completed.

On the other hand, this embodiment makes it possible to calculate at least one respective additional estimation for each characteristic heartbeat time with a higher reliability. These additional estimations can be used to improve the two real time detectors or a process, which generates a respiratory signal from the sum signal or from a sum signal, by applying a learning process in the continuous operation.

According to the embodiment just described, the first real time detector delivers the first real time detection result and the second real time detector delivers the second real time detection result in the calculation period. In one embodiment, the two real time detectors analyze the same sum signal but apply different methods of analysis. In a preferred alternative embodiment, the first real time detector analyzes a first sum signal and the second real time detector analyzes a second sum signal. These two sum signals differ from one another. The first sum signal is preferably generated from measured values of a first sensor array, and the second sum signal is generated from measured values of a second sensor array. It is also possible that the two sum signals are generated by applying different methods for processing measured values.

According to the present invention, the sum signal or each sum signal comprises a respective superimposition of a respiratory signal with a cardiogenic signal. The respiratory signal correlates with the intrinsic breathing activity of the patient, and the cardiogenic signal correlates with the cardiac activity of the patient. According to the embodiment just described, a first real time detector calculates a respective first real time estimation for each characteristic heartbeat time and uses a sum signal for this. A second real time detector calculates a second real time estimation and likewise uses a sum signal for this. The sum signal, which the first real time detector uses, may be the same sum signal as the sum signal, which the second real time detector uses, or be a different sum signal.

In one application of the present invention with the two detectors, a first sum signal and a second sum signal are generated and used. The first detector uses the first sum signal, and the second detector uses the second sum signal. These two sum signals stem from the same patient but are different from one another. The signal processing unit preferably generates
the first sum signal using measured values of a first sensor array and
the second sum signal using measured values of a second sensor array.

The signal processing unit calculates a first estimation and a second estimation for the respiratory signal, wherein the respiratory signal correlates with the intrinsic breathing activity of the patient and is comprised by the sum signal or by each sum signal. In order to calculate these estimations for one and the same respiratory signal, the signal processing unit uses the same respective estimation for each characteristic heartbeat time. This estimation was calculated beforehand by the signal processing unit, wherein the signal processing unit according to the present invention has used the two detection results of the two detectors, i.e., the two estimations for the same characteristic heartbeat time. To calculate the first estimation for the respiratory signal, the signal processing unit uses the estimated characteristic heartbeat time per heartbeat and, moreover, the first sum signal. For the second estimation, the signal processing unit uses the same estimated characteristic heartbeat time per heartbeat and, in addition, the second sum signal. The signal processing unit preferably calculates the first estimation and the second estimation for the respiratory signal by the signal processing unit approximately compensating by calculation the effect of the cardiogenic signal in the first sum signal or in the second sum signal by using the characteristic heartbeat times.

The two just described embodiments of the present invention can be combined with one another. The first just described embodiment provides that the first real time detector delivers the first real time detection result and the second real time detector delivers the second real time detection result in the calculation period. In the calculation period, the signal processing unit calculates a respective real time estimation for each characteristic heartbeat time and uses for this the two real time detection results. According to the second embodiment, the signal processing unit generates two different sum signals and calculates two estimations for the same respiratory signal and uses the same respective estimation for each characteristic heartbeat time for both estimations of the respiratory signal. The signal processing unit calculates
the first estimation for the respiratory signal using the first sum signal and using a respective estimation for each characteristic heartbeat time, and
the second estimation for the respiratory signal using the second sum signal and using the same estimation for the characteristic heartbeat time.

According to the combination, the signal processing unit uses a respective real time estimation for each characteristic heartbeat time to calculate the two estimations for the respiratory signal. As a result, the two estimations for the same respiratory signal are present in the calculation period, wherein these two estimations are based on the two sum signals.

The two estimations for the respiratory signal are preferably combined (merged) into a single estimation. In one application of this preferred embodiment, this combined (merged) estimation is used to actuate a ventilator. For example, a first signal quality index for the first estimation and a second signal quality index for the second estimation are used for the positioning. A signal quality index indicates how reliable the respective estimation is. Quality indicators of the estimations of the characteristic heartbeat times are used to calculate the signal quality index for the estimation of a respiratory signal.

According to the just described embodiment with the two real time detectors, an additional first detector calculates a respective additional first detection result for each characteristic heartbeat time. An additional second detector calculates a respective additional second detection result. These two additional detection results are not necessarily present in the calculation period. The signal processing unit calculates with higher reliability a respective additional estimation for each characteristic heartbeat time, for which the signal processing unit uses the two additional detection results and preferably has available a longer segment of the sum signal as well as more computing time. For example, each additional detector uses a sum signal segment for the complete heartbeat time period. After N heartbeats, N additional estimations, which have been obtained with higher reliability, are thus present. In this case, N>1 is a predefined number. The signal processing unit preferably uses these N additional estimations in order to correct by calculation future real time detection results of the first real time detector and of the second real time detector. By using the estimations, the signal processing unit calculates a first statistical deviation indicator and a second statistical deviation indicator. The first statistical deviation indicator is a statistical indicator of the deviation between the N first real time detection results and the N first estimations obtained with higher reliability. Correspondingly, the second statistical deviation indicator is a statistical indicator of the deviation between the N second real time detection results and the N second estimations obtained with higher reliability. The signal processing unit corrects by calculation the first real time detector and the second real time detector by using the first statistical deviation indicator and the second statistical deviation indicator, respectively. The deviation indicator is, for example, an offset, i.e., a systematic time shift between the real time detection result and the actual characteristic heartbeat time. After this correction by calculation, each real time detector delivers more reliable results in many cases.

This correction can be carried out continuously with the respective last N additional estimations. It is possible to correct by calculation an additional detector instead of or in addition to a real time detector.

According to the present invention, each first detector calculates a first detection result, and each second detector calculates a second detection result. In one embodiment, each detector additionally calculates a respective quality indicator, which is an indicator of the reliability, with which the detection result of this detector coincides with the actual characteristic heartbeat time. The signal processing unit calculates the estimation for a characteristic heartbeat time, for which the signal processing unit uses both the detection results for this characteristic heartbeat time and the quality indicators according to this embodiment according to the present invention. The detection results for a characteristic heartbeat time are thus combined into the estimation by using the quality indicators.

It is possible that a detector delivers different quality indicators for a plurality of consecutive heartbeats. One quality indicator therefore preferably always pertains to a single heartbeat or to a sequence of heartbeats.

In a variant of this embodiment, the signal processing unit uses the detection result, which has the greatest quality indicator, as an estimation for a characteristic heartbeat time. In another variant of this embodiment, the signal processing unit forms a weighted mean among a plurality of detection results, and it uses the quality indicators as weighting factors.

According to this embodiment, the quality indicators are used to fuse (combine) a plurality of detection results for one and the same heartbeat into an estimation of the characteristic heartbeat time of this heartbeat.

In one application of the present invention, the estimations for the characteristic heartbeat times are used to calculate at least one estimation for the respiratory signal. This respiratory signal correlates with the intrinsic breathing activity of the patient. The sum signal or each sum signal generated is a superimposition of the respiratory signal with the cardiogenic signal and optionally with unwanted signals. According to this application, the characteristic heartbeat times are used as follows: In the sum signal or in at least one sum signal, which the sensor array sends and which the signal processing unit receives, the contribution of the cardiogenic signal to the sum signal is compensated by calculation.

The estimation for the respiratory signal is preferably used to synchronize the ventilation strokes of a ventilator with the intrinsic breathing activity of a patient, especially with regard to the beginning and the end of a ventilation stroke as well as the amplitude and/or the frequency of the ventilation strokes. The respective start and the respective end of each inhalation effort of the patient are especially preferably detected by analyzing the estimation or at least one estimation for the respiratory signal, and the ventilation strokes are triggered with the goal that the respective beginning of each inhalation effort triggers a ventilation stroke and the respective end again ends this ventilation stroke. An inhalation effort may lead to a breath or even not bring about a breath. A gas mixture containing oxygen is fed to the patient during each ventilation stroke. This gas mixture additionally contains at least one anesthetic in one application, so that the patient is sedated or even anesthetized.

The process according to the present invention and the signal processing unit according to the present invention deliver a sequence of estimated characteristic heartbeat times. This sequence is used to determine the minimal or maximum heartbeat rate in one application of the present invention. In another application, the sequence is used to determine the temporal variability of the cardiac activity of the patient. For example, the time course (time series) of the heartbeat rate is calculated. The method of analysis, for example, a statistical method, is applied to the time course of the heartbeat times and optionally to that time course of the heartbeat rate. Such methods have become known by the term heart rate variability (HRV).

The present invention will be described below on the basis of an exemplary embodiment. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a view showing an exemplary segment of a cardiogenic signal in the course of a single heartbeat;

FIG. 2 is a schematic view showing which sensors/sensor arrays measure which different variables, which are used for the determination of an estimated respiratory signal;

FIG. 3 is an exemplary view showing an exemplary course of the sum signal as well as two heartbeat times and two heartbeat time periods;

FIG. 4 is a view showing three exemplary courses of a cardiogenic signal;

FIG. 5 is a schematic view showing the two function blocks for determining the estimated respiratory signal;

FIG. 6 is an exemplary view showing a reference signal segment that is generated and used;

FIG. 7 is a view showing an exemplary course of the compensation signal and two exemplary heartbeat time periods;

FIG. 8 is an exemplary schematic view showing heart time representations calculated based on five sum signals and a ventilator actuated by means of two sum signals;

FIGS. 9 (1/2) and 9(2/2) is an exemplary schematic view showing signals that are combined (merged) from four channels; and

FIG. 10 is an exemplary schematic view showing signals from two channels that are combined in real time.

### DESCRIPTION OF PREFERRED EMBODIMENTS

In the exemplary embodiment, the present invention is used for the ventilation and/or the automatic analysis of the vital parameters of a patient.

“Signal” shall be defined below as the course in the time range or even in the frequency range of a directly or indirectly measurable and temporally variable variable which correlates with a physical variable (a physical quantity that varies). In the present case, this physical variable is connected with the cardiac activity and/or with the intrinsic breathing activity and/or with the other muscle activity of a patient and/or with the ventilation of the patient and is generated by at least one signal source in the body of the patient and/or by a ventilator. A “respiratory signal” correlates with the intrinsic breathing activity of the patient, a “cardiogenic signal” correlates with the cardiac activity of the patient. A segment of this signal which pertains to a defined period is designated below as “signal segment.” The value of a signal at a defined time is designated as a signal value or even as a signal segment value.

In the exemplary embodiment, the present invention is used to automatically determine an estimation (representation) Sig_{res.est} for an electrical respiratory signal Sigᵣₑₛ, wherein the respiratory signal Sigᵣₑₛ to be estimated (represented) correlates with the intrinsic breathing activity of a patient P. This intrinsic breathing activity may be triggered by electrical pulses in the body of the patient, which the patient himself/herself generates, i.e., it may be a spontaneous breathing, or may be stimulated from outside. In both cases, the diaphragm muscles of the patient P accomplish this intrinsic breathing activity. This distinguishes the intrinsic breathing activity from a ventilation, which is caused by ventilation strokes of a ventilator. The subscript est indicates that the signal is estimated (is a representation of the signal).

In one application of the exemplary embodiment, the patient P is ventilated mechanically at least from time to time, while the estimated respiratory signal Sig_{res.est} is determined. The present invention is used in another application to monitor the patient P and to use the respiratory signal Sigᵣₑₛ to be estimated for this purpose, without the patient P being necessarily ventilated mechanically in this case.

Both the respiratory signal Sigres and the determined estimation Sig_{res.est} are variable over time, i.e., Sigᵣₑₛ=Sigᵣₑₛ(t) and Sig_{res.est}=Sig_{res.est}(t).

This respiratory signal Sigᵣₑₛ cannot be measured directly. On the one hand, the pulses generated in the body of the patient, which “actuate” the respiratory muscles, cannot be measured directly especially when electrodes pick up measured values on the skin of the patient, but only electrical pulses, which are generated during the contraction of the muscle fibers of the respiratory muscles. In addition, the electrical pulses, which cause the intrinsic breathing activity of the patient P, are superimposed by electrical pulses, which cause the cardiac activity of the patient P; more precisely: which are formed during the contraction of the heart muscles. Therefore, after a corresponding processing of measured values, only a sum signal Sigₛᵤₘ can be measured directly. This sum signal Sig_{Sum} is formed from a superimposition of the respiratory signal Sigᵣₑₛ being sought, which correlates with the intrinsic breathing activity of the patient, and of a cardiogenic signal Sigₖₐᵣ, which correlates with the cardiac activity.

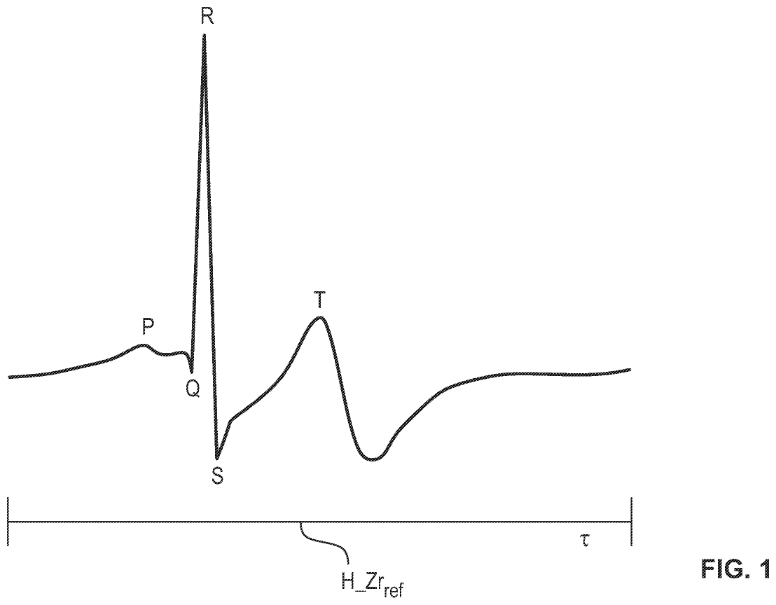

A typical segment of an electrically measured cardiogenic signal Sigₖₐᵣ in the course of a single heartbeat is shown in FIG. 1. A reference heartbeat time period H_Zr_{ref} is shown on the x axis, and the signal value is shown on the y axis, for example, in millivolts. Five peaks P, Q, R, S and T can be seen. The characteristic heartbeat time is, for example, the R peak or the temporal center between the Q peak and the S peak of this heartbeat.

The sum signal Sig_{Sum} is formed from the superimposition of the respiratory signal Sigᵣₑₛ with the cardiogenic signal Sigkar. As a rule, the sum signal Sig_{Sum} is additionally superimposed by unwanted signals.

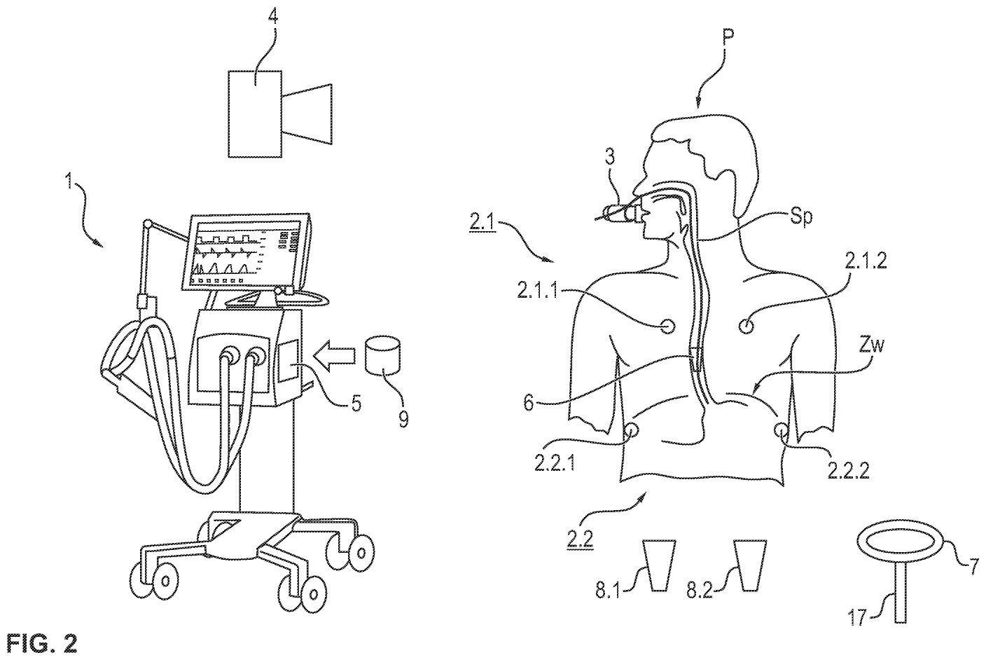

FIG. 2 schematically shows which signals can be generated from measured values by the measured values being processed automatically in a suitable manner. Shown are
the mechanically ventilated patient P,
the trachea Sp and the diaphragm Zw of the patient P,
a ventilator 1, which mechanically ventilates the patient Pat least from time to time and which comprises a data-processing signal processing unit 5, wherein the signal processing unit 5 has reading access and writing access to a memory 9 at least from time to time,
an intercostal pair 2.1 with two measuring electrodes 2.1.1 and 2.1.2, which are arranged on the right and on the left of the sternum and between two respective ribs of the patient P, i.e., in an area located near the heart,
a pair 2.2 located near the diaphragm with two measuring electrodes 2.2.1 and 2.2.2, which are arranged near the diaphragm of the patient P,
an electrode for ground, not shown,
a pneumatic sensor 3, which is located at a distance in space from the body of the patient P and comprises a measured value transducer, which is arranged, for example, in front of the mouth of the patient P, as well as an analysis unit, which may be arranged in the ventilator 1,
an optional sensor 4, which comprises an image recording device and an image analysis unit and is directed toward the chest area of the patient P,
an optional pneumatic sensor 6 in the form of a probe or a balloon in the trachea Sp and near the diaphragm Zw of the patient P,
a cuff 7 around a wrist of the patient P, wherein this cuff 7 holds a catheter 17 for measuring the time course of the blood pressure invasively,
two finger clips 8.1, 8.2, which are placed over a respective finger of the patient P or are positioned at a different location on the skin of the patient P, wherein the one finger clips 8.1 measures the degree of saturation of the blood with oxygen non-invasively, preferably by means of a plethysmographic method, and the other finger clip 8.2 measures the blood pressure of the patient P non-invasively, and
optionally electrodes, not shown, in the trachea of the patient P.

The intercostal pair 2.1 and the ground electrode deliver a first sum signal Sig_{Sum}[1] after signal processing. The pair 2.2 located near the diaphragm and the ground electrode deliver a second sum signal Sig_{Sum}[2] after signal processing. The additional sensors described above may deliver additional sum signals Sigₛᵤₘ[n], n >=3. It is also possible that the same sensor array delivers two different sum signals, for example, by applying different measuring methods. Such a sensor array delivering two different sum signals is described, for example, in DE 10 2009 035 018 A1 (corresponding U.S. US201 1028819 A1 is incorporated herein by reference). “The sum signal Sig_{Sum}” is discussed briefly below.

The signal processing preferably comprises a so-called baseline filtering. This process comprises the step that an average course is determined from the raw signal Sigmw, for example, by means of a statistical method, and the average course is subtracted from the raw signal Sig_{raw}. A course with isoelectric points is used in one application instead of the average course.

For example, a sum signal Sig_{Sum} in the form of a mechanomyogram (MMG signal) can also be generated and used instead of an electrical signal (EMG signal). Only the EMG sensors or MMG sensors are needed for the exemplary embodiment. It is also possible to generate such a signal as a sum signal Sig_{Sum}, which signal correlates with the time course of the change in the blood volume in the body of the patient, for example, by means of measured values, which are obtained by optical plethysmography.

The optical sensor 4 repeatedly measures a respective value for at least one anthropological parameter of the patient. The parameter is, for example, the current lung filling level and/or the current sitting posture of the patient P. The optical sensor 4 comprises, for example, a camera or other image recording device as well as an image analysis unit.

An indicator Pm, for the airway pressure and/or an indicator P. for the tracheal pressure can be generated from the measured values of the other sensors as well as of sensors, not shown, in the interior of the ventilator 1, and a pneumatic indicator Rum, which is likewise an indicator of the intrinsic breathing activity of the patient P, can be derived therefrom. According to a preferred embodiment, an estimation (representation) Sig_{res.est} for the electrical or mechanical respiratory signal Sigᵣₑₛ, on the one hand, and a pneumatic indicator Pₘᵤₛ, on the other hand, are determined. Thanks to this combination, the intrinsic breathing activity of the patient P is determined with higher reliability than in case of deriving only one signal. Furthermore, thanks to this combination it can in many cases be deduced how well the respiratory muscles of the patient P convert electrical pulses in the body of the patient P into pneumatic breathing activity (neuromechanical efficiency). The present invention can also be used in an embodiment, in which even the EMG signal or the MMG signal is generated, but not the pneumatic indicator P., for the breathing activity.

The estimated respiratory signal Sig_{res.est} determined according to the present invention is used, for example, for the following purposes:
A pneumatic indicator Pₘᵤₛ for the intrinsic breathing activity of the patient P is derived by using the respiratory signal Sig_{res.est}. The ventilation strokes, which the ventilator 1 brings about, are synchronized as well as possible with the intrinsic breathing activity of the patient P.
The neuromechanical efficiency of the breathing of the patient P is determined. This efficiency is an indicator of how well the respiratory muscles convert electrical signals into spontaneous breathing.
The state of the respiratory muscles of the patient P is determined (fatigue determination); the pneumatic indicator Pₘᵤₛ is not needed for this.
Asynchronies in the intrinsic breathing activity of the patient are detected; the pneumatic indicator Pmus is not needed for this as well.
In order to monitor the patient P, the estimated respiratory signal Sig_{res.est} and the respiratory EMG performance are determined and outputted as two vital parameters in a form perceptible to a person, preferably visually in the form of a respective time course, optionally together with the airway pressure P_{aw} or the tracheal pressure Pₑₛ.
It is possible that the patient P is not fully sedated, but rather the diaphragm muscles of the patient P carry out an intrinsic breathing activity. In this situation, an assist of the intrinsic breathing activity by a ventilation is triggered and/or carried out as a function of the estimated respiratory signal Sig_{res.est}. The ventilation strokes of the ventilation are preferably carried out as a function of the estimated (represented) respiratory signal Sig_{res.est}. For example, the ventilator I triggers the ventilation strokes as a function of the estimated respiratory signal Sig_{res.est} and/or ends the ventilation strokes and/or sets the respective amplitude of each ventilation stroke and/or the temporally variable frequency of the Is ventilation strokes as a function of the estimated respiratory signal Sig_{res.est}. The duration and the end of a ventilation stroke may also be automatically regulated as a function of the estimated respiratory signal Sig_{res.est}.

In order to regulate the ventilator 1 during the ventilation of the patient P or to monitor the patient P and to use the estimated respiratory signal Sig_{res.est} for the regulation or monitoring, the estimated respiratory signal Sig_{res.est} is determined with a high sampling frequency and/or with a short calculation period, i.e., the signal processing unit 5 delivers a new signal value Sig_{res.est}(t) at each sampling time t. “High sampling frequency” is defined as an interval of less than 5 [msec], and preferably less than 3 msec located between two consecutive sampling times. “Short calculation time” is defined as a calculation time of less than 5 [msec], preferably less than 3 msec. In particular, the sampling frequency is preferably at least 1 kHz, especially preferably at least 2 kHz for the fatigue determination. Some steps of the process described below are, by contrast, carried out in the exemplary embodiment with a low sampling frequency, namely with a frequency that is in the range of the heartbeat rate, i.e., between 1 Hz and 2 Hz. The calculation time is long in another embodiment.

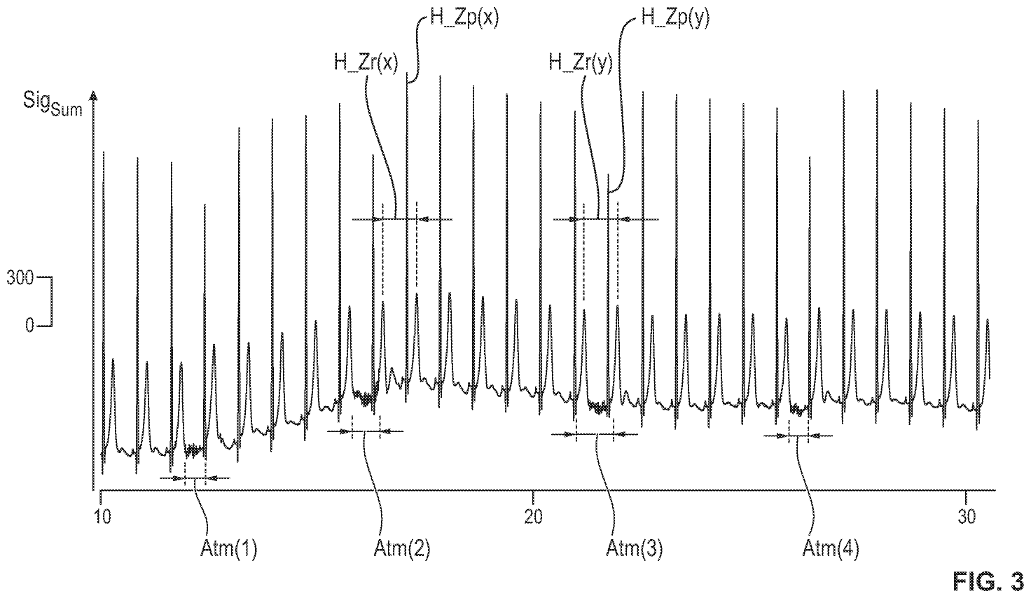

FIG. 3 shows an exemplary time course of a sum signal Sig_{Sum}[.] The segment shown in FIG. 3 comprises four breaths and a plurality of heartbeats. Shown are four periods Atm(1), . . . , Atm(4) of the four breaths and for the two exemplary heartbeats x and y a respective heartbeat time period H_Zp(x) and H_Zp(y) and a characteristic heartbeat time H_Zp(x) and H_Zp(y). It can be seen that the cardiogenic signal Sigₖₐᵣ in one heartbeat time period is many times greater than the respiratory signal Sigᵣₑₛ in this period. Outside of the heartbeat time period H_Zr(x), H_Zr(y), the respiratory signal Sigᵣₑₛ is, however, sufficiently strong compared to the cardiogenic signal Sigₖₐᵣ and may therefore be determined from the sum signal Sig_{Sum}, for example, by the sum signal Sig_{Sum} being used as the estimated respiratory signal Sig_{res.est} outside of each heartbeat time period.

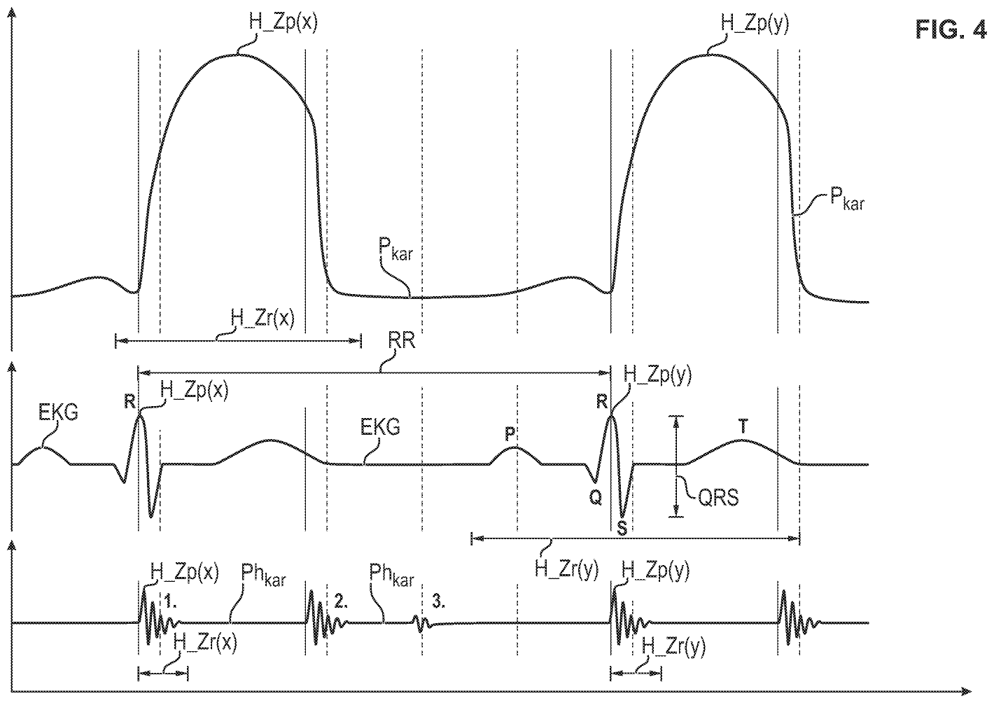

FIG. 4 shows in an exemplary manner three typical time courses of the cardiogenic signal Sigₖₐᵣ, namely from top to bottom
a pneumatic signal Pₖₐᵣ for the ventricular pressure, measured in mmHg,
an electrical ECG signal, measured in millivolts, as well as
an acoustic signal Phkar for the heart sounds, measured with an acoustic sensor.

The x axis applies to all three courses. The y axes pertain to the respective mass unit of the signal. The time t is plotted on the x axis; the respective value of the cardiogenic signal Sigₖₐᵣ is plotted on the y axis. The period shown overlaps two consecutive heartbeats x and y. In case of the ECG signal, each heartbeat comprises a so-called P wave, a QRS phase and a T wave.

The respective heartbeat time period H_Zr(x) or H_Zr(y) as well as the characteristic heartbeat times HZp(x) and H_Zp(y) of the two exemplary heartbeats No. x and No. y are shown for each time course. For example, the R peak is used as a characteristic time HZp(x) of a heartbeat in case of the ECG signal. The interval RR between two consecutive heartbeats as well as the QRS amplitude QRS of a heartbeat are shown in FIG. 4. As is suggested in FIG. 4, the cardiogenic signal Sigₖₐᵣ is three orders of magnitude greater than the respiratory signal Sigᵣₑₛ in the area of the P wave up to the T wave of a heartbeat and equal or smaller in the remaining area. As can, furthermore, be seen in FIG. 4, how and especially with what length a heartbeat time period and a characteristic heartbeat time H_Zp(x), H_Zp(y), . . . are specified depends on the signal used. Different possible specifications are shown in FIG. 4.

The sum signal or each sum signal Sig_{Sum} is a superimposition of the respiratory signal Sigᵣₑₛ being sought and of the cardiogenic signal Sigₖₐᵣ as well as optionally of unwanted signals. In one application of the present invention, the characteristic heartbeat time H_Zp(x) of each heartbeat x is used to compensate by calculation the effect of the cardiogenic signal Sigₖₐᵣ on a sum signal Sigₛᵤₘ.

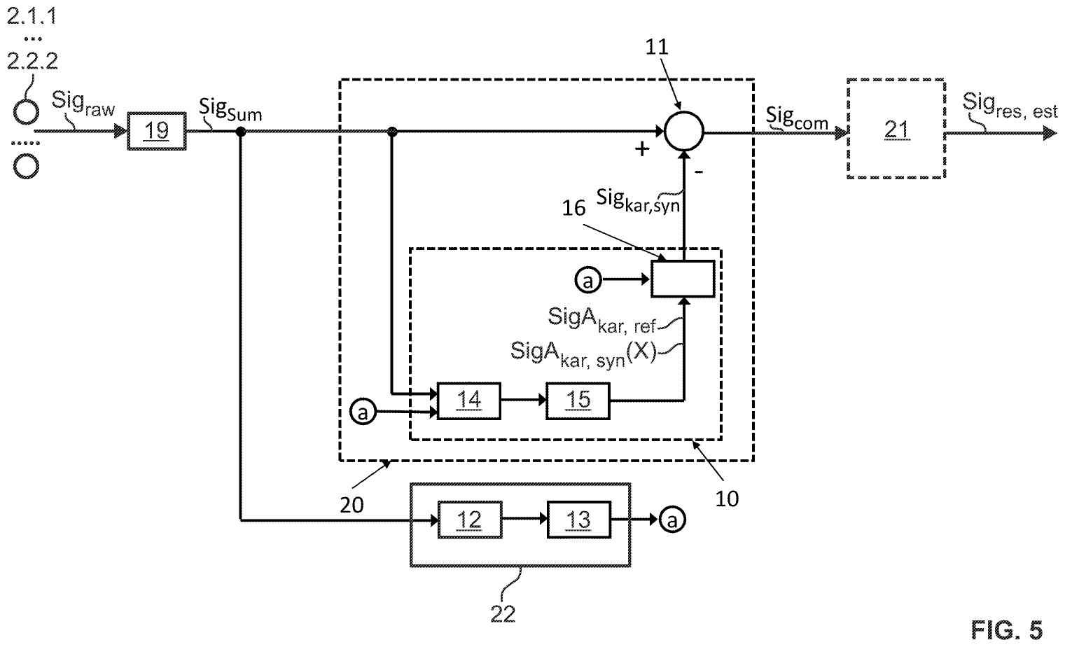

FIG. 5 schematically shows a measured value processor 19. This measured value processor 19 processes the raw signal Sigiaw, which is delivered by the sensors 2.1.1 through 2.2.2 after a signal amplification. The measured value processor 19 eliminates by calculation low-frequency vibrations, standardizes the raw signal Sig_{raw}, for example, by means of the above-described baseline filtering, and delivers the sum signal Sig_{Sum}.

FIG. 5 schematically shows, furthermore, a function block 20, which receives the sum signal Sig_{Sum} and carries out the just mentioned compensation by calculation of the cardiogenic signal Sigₖₐᵣ. This function block 20 carries out different signal processing steps in order to eliminate by calculation the cardiogenic signal Sigₖₐᵣ from a sum signal Sig_{Sum}, i.e., to compensate at least partially the effect of the cardiac activity on a measured sum signal Sig_{Sum}. For each heartbeat x, the function block 20 detects in the sum signal Sig_{Sum} a sum signal segment SigA_{Sum}(x), in which the heartbeat x takes place. Each sum signal segment SigAₛᵤₘ(x) pertains to the same reference heartbeat time period H_Zr_{ref}, cf. FIG. 1. In the heartbeat time period H_Zr(x), the sum signal Sig_{Sum} is almost exclusively determined by the cardiogenic signal Sigₖₐᵣ, so that the remaining signal components can be ignored. The function block 20 delivers a compensation signal Sig_{com}.

A functional unit 11 of the compensation function block 20 generates a synthetic cardiogenic signal Sig_{kar.syn}, which is an approximation (estimation) for the cardiogenic signal Sigₖₐᵣ and is composed of signal segments. The function unit 11 compensates by calculation the contribution of the cardiogenic signal Sigₖₐᵣ to the sum signal Sig_{Sum}, for example, by subtraction of the synthetic cardiogenic signal Sig_{kar.syn}, and generates the compensation signal Sig_{com} as a result.

Exemplary methods for generating such a compensation signal Sig_{com} are described in the following publications, which are incorporated herein by reference:
M. Ungureanu and W. M. Wolf: “Basic Aspects Concerning the Event-Synchronous Interference Canceller,” IEEE Transactions on Biomedical Engineering, Vol. 53, No. 11 (2006), pp. 2240-2247;
L. Kahl and U. G. Hofmann: “Removal of ECG artifacts from EMG signals with different artifact magnitudes by template subtraction,” Current Directions in Biomedical Engineering, 2019; 5(1), pp. 357-360;
DE 10 2007 062 214 B3 (corresponding to U.S. Pat. No. 8,109,269 B2),
EP 3 381 354 A1; and
the subsequently published patent disclosure DE 10 2019 006 866 A1.

In an embodiment the compensation function block 20 applies one of the methods described in the above-mentioned publications.

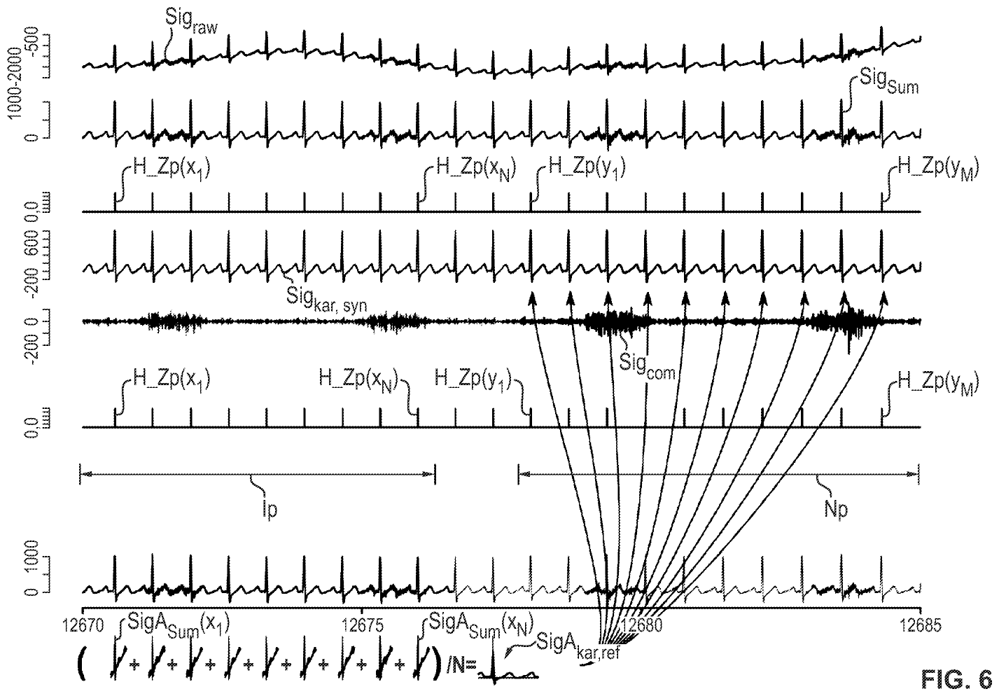

The compensation function block 20 generates in an initialization phase Ip a cardiogenic reference signal segment SigA_{kar.ref}, which is valid for this patient P in this current situation and which is stored in the memory 9, and applies this signal segment again to each heartbeat in a subsequent use phase. The initialization phase Ip is preferably repeated continuously, for which the respective last N heartbeats are used. As a result, the reference signal segment SigA_{kar.ref} is updated continuously and is especially adapted to an altered state of the patient P. N is preferably between 50 and 100. The value N=9 is used in FIG. 6.

The following steps are carried out in both phases Ip, Np:
A functional unit 12 identifies the heartbeat time period H_Zr(x) of each heartbeat x in the sum signal Sig_{Sum}, preferably the respective beginning, e.g., the P peak, and the respective end, e.g., the T peak, and/or the respective QRS phase of each heartbeat x.
A functional unit 13 determines the respective characteristic heartbeat time H_Zp(x) of each heartbeat, especially preferably with a tolerance of a few msec. The tolerance is especially preferably at most half of the period between two consecutive sampling times for the determination of the sum signal Sig_{Sum}, wherein this period is preferably below 1 msec.
The function block 22 with the functional units 12 and 13 needs a plurality of values of the sum signal Sig_{Sum} for a plurality of consecutive sampling times to determine the characteristic heartbeat time H_Zp(x) of each heartbeat with sufficient accuracy. What consequences this has, for example, for the actuation of the ventilator 1 is explained below.

Furthermore, the following steps are carried out in the initialization phase Ip:
A sum signal segment SigA_{Sum}(x) of the sum signal Sig_{Sum} belongs to each heartbeat No. x, cf. FIG. 3 and FIG. 4. A functional unit 14 superimposes by calculation the N sum signal segments SigAₛᵤₘ(x₁), . . . , SigA_{Sum}(x_{N}) for the last N heartbeats x₁, . . . , x_{N}. As needed, these sum signal segments SigA_{Sum}(x₁), . . . , SigA_{Sum}(x_{N}) are cut, compressed or stretched to a consistent length. A process for superimposition of segments is described in M. Ungureanu and W. M. Wolf, mentioned above. The N sum signal segments SigA_{Sum}(x₁), SigA_{Sum}(x_{N}) for the N heartbeats are preferably superimposed such that the N sum signal segments have the same length and the R peaks are located above one another. Each sum signal segment SigA_{Sum}(x) thus pertains to the same reference heartbeat time period H_Zr_{ref}. A relative time in this reference heartbeat time period H_Zr_{ref} is designated by τ. A relative time τ=τ(t) in this relative heartbeat time period corresponds to each absolute time t of the sum signal segment SigA_{Sum}(x). Instead of the designation “relative time,” the designation “heart phase ϕ” with a value range from 0° to 360° or from 0 to 2 π can be used.
A functional unit 15 generates a cardiogenic reference signal segment (template) SigA_{kar,ref} from the superimposition of N sum signal segments SigA_{Sum}(x₁), SigA_{Sum}(x_{N}), which the functional unit 14 has generated. This cardiogenic reference signal segment SigA_{kar.ref} describes approximately the course of the cardiogenic signal Sigₖₐᵣ during a single heartbeat and likewise pertains to the reference heartbeat time period H_Zr_{ref}. The characteristic heartbeat time H_Zp(x) is preferably τ=0. As was already mentioned, the cardiogenic component in the sum signal Sig_{Sum} is many times greater than the respiratory component during a heartbeat, and the respiratory components during a heartbeat are largely “averaged out” by averaging over N sum signal segments. The functional unit 15 preferably applies a learning method to the N sum signal segments SigA_{Sum}(x₁), SigA_{Sum}(x_{N}). The cardiogenic reference signal segment SigA_{kar.ref} is preferably stored in the memory 9.

The following steps are carried out in the use phase Np:
The functional unit 13 detects in the sum signal Sigₛᵤₘ the heartbeats and determines the respective characteristic heartbeat time H_Zp(x) of each heartbeat x detected.
For each heartbeat x, the cardiogenic reference signal segment SigA_{kar.ref} is used again. In one embodiment, this is subtracted unchanged from the sum signal segment SigA_{kar.rer} (template subtraction). The functional unit 16 carries out this step.
By contrast, the functional unit 16 optionally additionally uses the value of at least one anthropological parameter, which influences the cardiac activity and thus the cardiogenic signal Sigₖₐᵣ and has been measured in case of this heartbeat No. x. The lung filling level and an indicator of the current posture of the patient P as well as the interval RR between the R peaks of two consecutive heartbeats are examples of such an anthropological parameter. The functional unit 16 adapts for each heartbeat the cardiogenic reference signal segment SigA_{kar,ref} to the parameter value or each parameter value measured during this heartbeat x and as a result generates a cardiogenic signal segment SigA_{kar.syn}(x).
The functional unit 16 positions the cardiogenic reference signal segment SigA_{kar.ref} or optionally the adapted cardiogenic signal segment SigA_{kar.syn}(x) of the current heartbeat in a correctly timed manner, e.g., in a QRS-synchronized manner. As a result, a new synchronized segment of the synthetic cardiogenic signal Sig_{kar.syn} is generated. The synthetic cardiogenic signal Sig_{kar.syn} is preferably outputted in a form perceptible by a person.
A functional unit 11 compensates in the newest sum signal segment SigA_{Sum}(x) the effect of the cardiogenic signal Sigₖₐᵣ, for example, by subtracting the cardiogcnic reference signal segment SigA_{kar.ref} or the adapted cardiogenic signal segment SigA_{kar.syn}(x) from the newest sum signal segment SigA_{Sum}(x).

A preferred embodiment for applying a learning method in the initialization phase Ip as well as the respective value of an anthropological parameter in the use phase for each heartbeat is described in the subsequently published patent disclosure DE 10 2019 006 866 A1 mentioned above.

As the beginning of the process, i.e., after the patient P is connected to the measuring electrodes 2.1.1 through 2.2.2, the initialization phase Ip is carried out, which comprises a time period of N heartbeats. This initialization phase Ip is preferably carried out again, especially with the respective last N heartbeats. In this initialization phase Ip, the compensation function block 20 generates, as described above, an initial cardiogenic reference signal segment SigAkar.me as a function of the sum signal segments SigA_{Sum}(x₁), SigA_{Sum}(x_{N}) for the last N heartbeats.

During the process, i.e., in the use phase Np, the compensation function block 20 adapts the cardiogenic reference signal segment SigA_{kar.ref} to the respective last N heartbeats, i.e., to the last N sum signal segments SigA_{Sum}(x₁), SigA_{Sum}(x_{N}) and stores the cardiogenic reference signal segment in the memory 9. The steps in the initialization phase Ip and the adaptation to the respective last N heartbeats are carried out with the low sampling frequency, which is approximately equal to the heartbeat rate.

The N sum signal segments for a respective heartbeat are preferably superimposed with the double time resolution of the sum signal Sig_{Sum}. This means: The values of the sum signal Sig_{Sum} are determined with a high sampling frequency, i.e., the interval Δt between two sampling times is 1/f. The time resolution is increased by calculation to, e.g., 2f or 3f, e.g., by a respective signal value Sig_{Sum}(t+Δt/2) being positioned by calculation, for example, by interpolation between two signal values Sig_{Sum}(t) and Sig_{Sum}(t+Δt) derived from measured values.

After the initialization phase Ip, the following steps are carried out with the high sampling frequency (a few msec or even only a few tenths of a msec):
The signal processing unit 5 derives from measured values a respective new value Sig_{Sum}(t) for the sum signal Sig_{Sum}.
The function block 22 with the functional units 12 and 13 detects in the sum signal Sig_{Sum} the beginning and the precise characteristic time H_Zp(x) of a heartbeat x and determines a new sum signal segment (SigA_{Sum}(x) as a result.
The compensation function block 20 optionally adapts the cardiogenic reference signal segment SigA_{kar.ref} to the respective value of at least one anthropological parameter, determines the associated relative time τ=τ(t) and generates an additional signal segment by positioning in a correctly timed manner, namely the temporally newest segment SigA_{kar.syn}(x) of the synthetic cardiogenic signal Sig_{kar.syn}.
The functional unit 11 subtracts from the new value Sig_{Sum}(t) the value SigA_{kar.ref}[τ(t)] or SigA_{kar.syn}(x)[τ(t)] of the cardiogenic reference signal segment SigA_{kar.ref} or the value of the adapted cardiogenic signal segment SigA_{kar.ref}(x) for the same relative time τ, i.e.,

Sig_{com}(t)=Sig_{Sum}(t)−SigAₖₐᵣ[τ(t)] or

Sig_{com}(t)=Sig_{Sum}(t)−SigA_{kar.syn}(x)[τ(t)].

It is also possible that the functional unit 11 compensates the cardiogenic effect in a different way, for example, by means of a high pass filter or by an independent component analysis or by a so-called blind source separation, which is described, for example, in DE 10 2015 015 296 A1.
The compensation function block 20 outputs a new signal segment SigA_{com}(x) for the compensation signal Sig_{com}.

The output signal Sig_{cum} of the compensation function block 20 is used in one embodiment as the estimated signal Sig_{res.est} for the respiratory signal Sigᵣₑₛ being sought. In another embodiment, the output signal is attenuated, and especially by an attenuation function block 21, cf. FIG. 5. The attenuation function block 21 preferably comprises a high pass filter with a limit frequency between 10 Hz and 50 Hz to remove low-frequency residues of the cardiogenic signal Sigₖₐᵣ. An exemplary embodiment of this attenuation function block 21 is described in the subsequently published patent disclosure DE 10 2020 002 572 A1 (corresponding publication US2021338176 (A1) is incorporated herein by reference).

FIG. 6 shows in an exemplary manner how a cardiogenic reference signal segment SigA_{kar,ref} is generated. The time in [sec] is plotted on the x axis, and the signal value in μV is plotted on the y axis. In addition, the initialization phase Ip and the use phase Np are shown. In the example shown, N=9.

The following signals are shown in FIG. 6 from top to bottom:
the raw signal Sig_{raw},
the sum signal Sigₛᵤₘ, which the measured value processor 19 generates from the raw signal Sig_{raw},
the sequence of the detected heartbeat times H_Zp(x₁), H_Zp(x_{N}), H_Zp(y₁), H_Zp(y_{M}), . . . ,
the compensation signal Sig_{com}, which is generated from the cardiogenic reference signal segment SigA_{kar,ref} by using the detected heartbeat times H_Zp(y₁), H_Zp(y_{M}),
again the sequence of the detected heartbeat times H_Zp(x₁), H_Zp(x_{N}), H_Zp(y₁),
the N sum signal segments SigA_{Sum}(x₁), SigA_{Sum}(x_{N}) for the N heartbeat periods H_Zr(x₁), H_Zr(x_{N}) of the initialization phase Ip, and
schematically how the arithmetic mean is formed over the N sum signal segments SigA_{Sum}(x₁), SigA_{Sum}(x_{N}) and is used as the cardiogenic reference signal segment SigA_{kar.ref}.

It can be seen that the raw signal Sig_{raw} has low-frequency oscillations, i.e., oscillations with a frequency lower than the heartbeat rate. In addition, the signal values are between −2000 μV and −500 μV. The sum signal Sig_{Sum} has signal values between 0 μV and 1000 μV and has no low-frequency oscillations.

In the subsequent use phase Np, the compensation signal Sigcom is used by using the cardiogenic reference signal segment SigA_{kar.ref}, and the heartbeat times H_Zp(y₁), H_Zp(y_{M}), . . . are calculated. As already explained, the cardiogenic reference signal segment SigA_{kar.ref} is preferably updated continuously, for which the respective last N sum signal segments SigA_{Sum}(x₁), SigA_{Sum}(x_{N}) are used.

The upper four signals are generated with a higher sampling frequency and/or in a shorter calculation period. The lower three signals are generated with a lower sampling frequency and/or in a longer calculation period and therefore, as a rule, with higher accuracy.

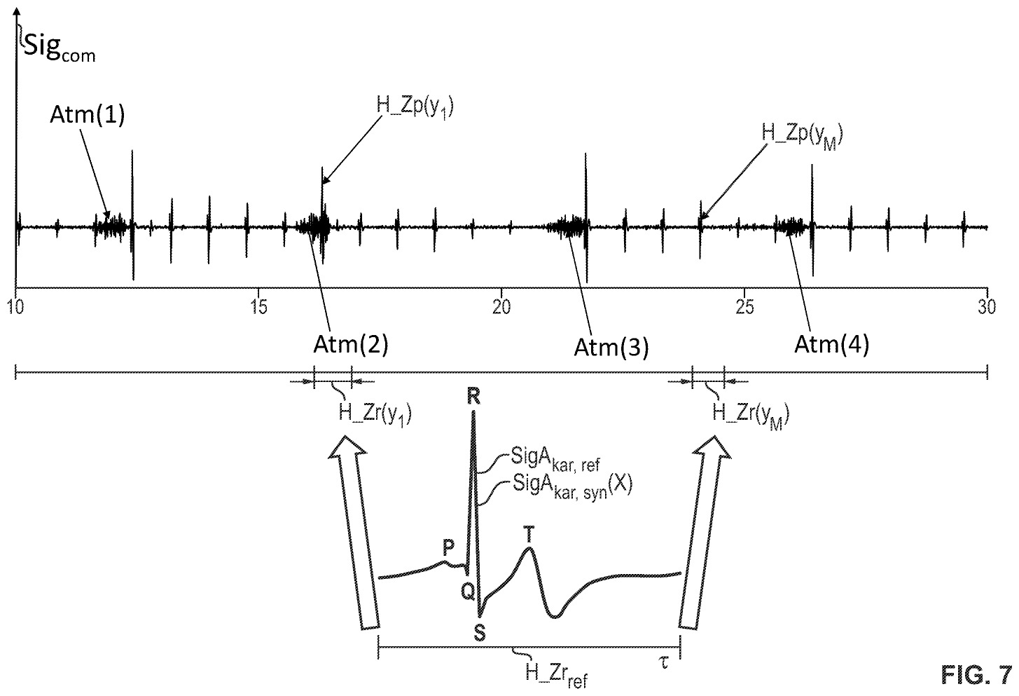

FIG. 7 shows at the top an exemplary course of the compensation signal Sig_{com}. This exemplary course is formed because the compensation function block 20 processes, as just described, the sum signal Sig_{Sum} shown as an example in FIG. 5 and FIG. 6. In addition, two exemplary heartbeat time periods H_Zp(y₁) and H_Zp(y_{M}) are shown in FIG. 7.

A reference heartbeat time period H_Zrn, is predefined. The time in the reference heartbeat time period H_Zr_{ref} is designated by τ. FIG. 7 shows how the same reference heartbeat time period H_Zr_{ref} is mapped to these two characteristic heartbeat times H_Zp(y₁) and H_Zp(y_{M}) one after the other. This mapping is necessary, so that the functional unit 11 can carry out the compensation shown in FIG. 5. The respective characteristic heartbeat time H_Zp(y₁), H_Zp(y_{M}) is needed for this mapping as well.

For example, if the estimated respiratory signal Sig_{res.est} is used for regulation of the ventilator 1, a new signal value is quasi needed in real time. The following problem additionally occurs in this case: The newest segment SigA_{kar.syn}(x) of the synthetic cardiogenic signal Sig_{kar.syn} may only be positioned in a correctly timed manner with sufficient accuracy if the characteristic heartbeat time H_Zp(x) has been detected. This is, as a rule, the case only if the R peak of this heartbeat has been detected, however. The newest sum signal segment SigA_{Sum}(x) cannot be positioned precisely in a correctly timed manner, but only estimated temporally in the period between the beginning of a heartbeat and the R peak of this heartbeat.

The process according to the present invention improves the temporal positioning by calculation of a heartbeat, and more precisely, the detection of each characteristic heartbeat time H_Zp(x). This positioning by calculation is also designated as QRS detection.

A heartbeat detection in real time is required for some applications. More precisely: A characteristic heartbeat time H_Zp(x) shall already be detected before the heartbeat time period H_Zr(x) of this heartbeat x is ended. Therefore, it is desired to detect the characteristic heartbeat time H_Zp(x) with an accuracy below 0.5 msec, preferably below 0.25 msec.

One idea of the process is to analyze the sum signals of different measuring channels, as well as by different methods, as a result of which a plurality of estimations for characteristic heartbeat points are determined for each period. Each estimation for a characteristic heartbeat time is, as a rule, provided with an unavoidable error. The quality of the estimation is evaluated with a quality indicator.

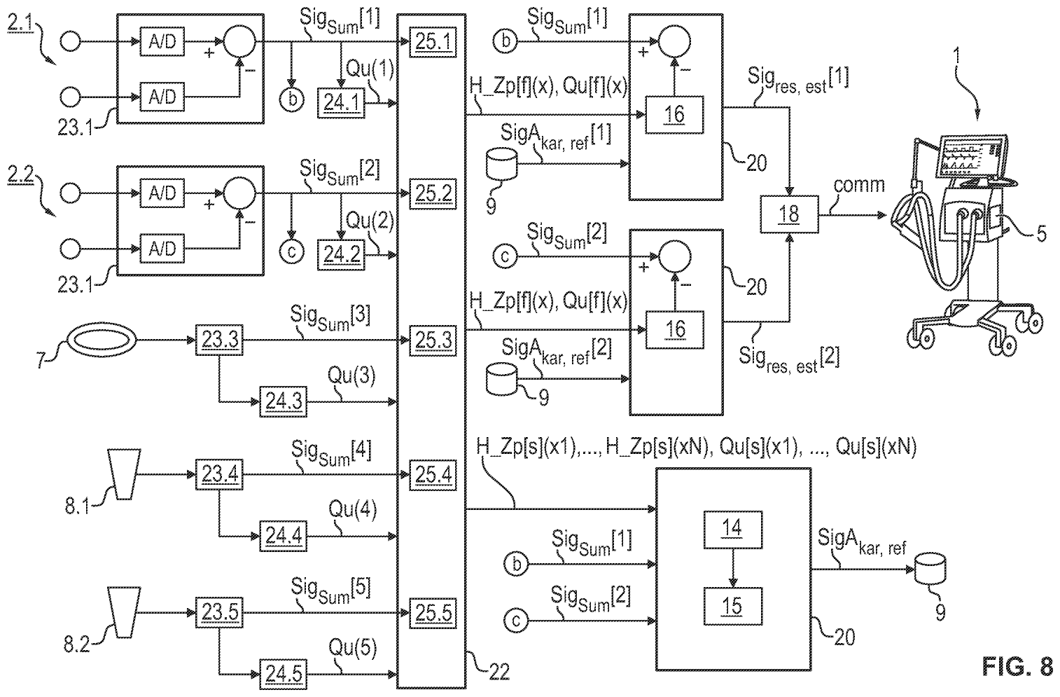

FIG. 8 shows one possible application of the present invention. In this application, the ventilator I shall be actuated in order to trigger ventilation strokes. The ventilation strokes shall be carried out in a manner synchronized with the intrinsic breathing activity of the patient P. A control device 18 generates control commands for the ventilator 1 and uses for this an estimation Sigres.est for the respiratory signal Sigᵣₑₛ.

In the exemplary embodiment, the control device 18 uses two different estimations Sig_{res,est}[1] and Sigre,est[2] for the respiratory signal Sigᵣₑₛ. The first estimation Sig_{res.est}[1] is based on measured values of the intercostal pair 2.1 of measuring electrodes, and the second estimation Sig_{res.est}[2] is based on measured values of the pair 2.2 of measuring electrodes located near the diaphragm. The intercostal pair of measuring electrodes 2.1 delivers a first sum signal Sig_{Sum}[1], from which the first estimation Sig_{res.est}[1] for the respiratory signal Sigᵣₑₛ is derived. The pair of measuring electrodes 2.2 located near the diaphragm delivers a second sum signal Sig_{Sum}[2], from which the second estimation Sig_{res.est}[2] for the respiratory signal Sigᵣₑₛ is derived. In practice, these two sum signals Sig_{Sum}[1] and Sig_{Sum}[2] differ from one another, among other things because of the different positions of the measuring electrodes 2.1.1 through 2.2.2 on the skin of the patient P and thus because of the different positions in relation to the signal sources (heart muscles and respiratory muscles). Ideally, the cardiac activity acts on both sum signals Sig_{Sum}[1] and Sig_{Sum}[2] simultaneously, often with different intensity.

The compensation function block 20, which was described with reference to FIG. 5, compensates by calculation the effect of the cardiac activity, i.e., the effect of the cardiogenic signal Sigₖₐᵣ, especially the effect on the first sum signal Sig_{Sum}[1], on the one hand, and the effect on the second sum signal Sig_{Sum}[2], on the other hand. By the compensation function block 20 compensating by calculation the effect of the cardiogenic signal Sigₖₐᵣ on the first sum signal Sig_{Sum}[1], the compensation function block 20 calculates the first estimation Sig_{res.est}[1] for the respiratory signal Sigᵣₑₛ. By the compensation function block 20 compensating by calculation the effect of the cardiogenic signal Sigₖₐᵣ on the second sum signal Sig_{Sum}[2], it calculates the first estimation Sig_{res.est}[2] for the respiratory signal Sigᵣₑₛ. Therefore, the compensation function block 20 is shown schematically twice in FIG. 8.

The compensation block 20 uses for the compensation by calculation, in addition, a cardiogenic reference signal segment for the sum signal Sig_{Sum}[1] and a cardiogenic reference signal segment for the sum signal Sig_{Sum}[2], i.e., a total of two cardiogenic reference signal segments SigA_{kar.ref}[1] and SigA_{kar.ref}[2]. In case three or even more different sum signals are used to derive a respective estimation for the respiratory signal, three or even more cardiogenic reference signal segments are also derived and used. These at least two reference signal segments are stored in the memory 9 and approximately describe the course of the cardiogenic signal Sigₖₐᵣ in the course of a single heartbeat. These cardiogenic reference signal segments SigA_{kar.ref}[1] and SigA_{kar.ref}[2] were preferably generated on the basis of the last N heartbeats, as this was described above. It is also possible to use two respective adapted signal segments SigA_{kar.syn}[1](x) and SigA_{kar.syn}[2](x) for each heartbeat.

The cardiogenic reference signal segment SigA_{kar.ref} must be positioned in a correctly timed manner to the first sum signal or the second sum signal. The function block 20 uses for this the respective characteristic heartbeat time H_Zp(x) of a heartbeat. Because the estimations Sig_{res.est}[1] and Sig_{res.est}[2] generated by the function block 20 are used to actuate the ventilator 1, estimations in real time are needed for these characteristic heartbeat times. In particular, a characteristic heartbeat time H_Zp(x) is needed before the heartbeat time period H_Zr(x) of this heartbeat has elapsed, i.e., before the heartbeat x is carried out completely.

The function block 22 calculates a respective estimation H_Zp[f](x) of the characteristic heartbeat time H_Zp(x) in real time for each heartbeat x. The abbreviation f means “fast.” This estimation H_Zp[f](x) is available sufficiently quickly, so that the function block 20 can approximately position the cardiogenic reference signal segment SigA_{kar.ref} in a correctly timed manner.

In the exemplary embodiment, the function block 22 uses signals from five different measuring channels, namely
the first sum signal Sig_{Sum}[1], which was generated on the basis of the intercostal pair of measuring electrodes 2.1,
the second sum signal Sig_{Sum}[2], which was generated on the basis of the pair of measuring electrodes 2.2 located near the diaphragm,
a third sum signal Sig_{Sum}[3], which was generated on the basis of measured values of the catheter 17 at the cuff 7 for the invasive measurement of the blood pressure,
a fourth sum signal Sig_{Sum}[4], which was generated on the basis of measured values of the first finger clip 8.1 for the non-invasive measurement of the oxygen saturation, and
a fifth sum signal Sig_{Sum}[5], which was generated on the basis of measured values of the second finger clip 8.2 for the non-invasive measurement of the blood pressure.

It is possible to obtain additional sum signals (not shown) by means of the following sensors:
with mechanomyographic (MMG) sensors,
by means of a process of electrical impedance tomography (EIT), for example, of an EIT belt around the lungs and/or around the chest of the patient P,
by means of the probe 6 in the trachea Sp, which measures the tracheal pressure Pₑₛ,
by means of a strain gauge at the chest of the patient P, and
by means of the optical sensor 4, which optimally measures the body of the patient P, cf. FIG. 2.

FIG. 8 shows five measured value processors, namely
a measured value processor 23.1 for the intercostal pair of measuring electrodes 2.1, wherein the measured value processor 23.1 delivers the first sum signal Sig_{Sum}[1],
a measured value processor 23.2 for the pair of measuring electrodes 2.2 located near the diaphragm, wherein the measured value processor 23.2 delivers the second sum signal Sig_{Sum}[2].
a measured value processor 23.3 for the cuff 7, wherein the measured value processor 23.3 delivers the third sum signal Sig_{Sum}[3],
a measured value processor 23.4 for the first sensor 8.1, wherein the first sensor 8.1 measures the oxygen saturation and wherein the measured value processor 23.4 delivers the fourth sum signal Sig_{Sum}[4], and
a measured value processor for the second sensor 8.2, wherein the second sensor 8.2 measures the oxygen saturation and wherein the measured value processor delivers the fifth sum signal Sig_{Sum}[5].

Ideally, all these five measuring channels 2.1 through 8.2 deliver the same characteristic heartbeat time H_Zp(x) for a heartbeat x, because all sum signals result from a superimposition of the same cardiac activity and the same breathing activity of the patient P. In practice, however, disturbance variables act in different ways on these five sum signals Sig_{Sum}[1] through Sig_{Sum}[5], especially because the five sensors 2.1 through 8.2 are arranged at different positions on the skin of the patient P. The sum signals Sig_{Sum}[3] through Sig_{Sum}[5] are used in the exemplary embodiment only for the detection of the characteristic heartbeat times, but, moreover, not for the estimation of the respiratory signal Sigᵣₑₛ.

The function block 22 applies up to five different detectors 25.1 through 25.5, wherein each detector pertains to a respective sum signal Sig_{Sum}[1] through Sig_{Sum}[5] and delivers an estimation (representation) for the actual characteristic heartbeat time H_Zp(x). Such an estimation is often also designated as QRS detection. As is suggested in FIG. 8, an estimation for the characteristic heartbeat time depends considerably on the sensor used. It is possible that a detector 25.1 through 25.5 does not detect individual heartbeats.

In one variation, the function block applies two detectors 25.1.f, 25.1.s to the first sum signal Sig_{Sum}[1] and two additional detectors 25.2.f, 25.2.s to the second sum signal Sig_{Sum}[2], wherein the two detectors 25.1.f and 25.2.f deliver a respective result (an estimation for a characteristic heartbeat time) H_Zp[1.f](x), H_Zp[2.f](x) in real time and the two other detectors 25.1.s and 25.2.s deliver a result H_Zp[1.s](x), H_Zp[2.s](x) only after the end of the QRS phase, but deliver this result with higher accuracy. In this embodiment as well, the function block 22 may apply at least one of the additional detectors 25.3, 25.4, 25.5.

Some sensors, especially non-electrical sensors, inevitably deliver a sum signal with delays. One example is the catheter 17, which is filled with a liquid and is held by the cuff 7. The catheter 17 measures the time course of the blood pressure invasively. The pressure propagates in the liquid in the catheter 17 approximately at the speed of sound. A time delay results from the propagation speed of the pressure in the catheter 17.

In one embodiment, the function block 22 uses only results from the two detectors 25.1.f and 25.21 These results pertain to the two sum signals Sig_{Sum}[1] and Sig_{Sum}[2], which were generated from measured values of the two pairs of measuring electrodes 2.1 and 2.2, wherein the two sum signals Sig_{Sum}[1] and Sig_{Sum}[2] deliver the two estimations H Zp[11](x) and H_Zp[2.f](x) to calculate the estimation H_Zp[f](x) in real time.

In addition, FIG. 8 shows five optional functional units 24.1 through 24.5, which each calculate a quality indicator Qu(1) through Qu(5). The respective quality indicator Qu(1) through Qu(5) indicates how well the characteristic heartbeat time can be detected in the respective sum signal Sig_{Sum}[1] through Sig_{Sum}[5]. The function block 22 additionally uses these five quality indicators Qu(1) through Qu(5) in one embodiment.

As was already explained, each detector 25.1 through 25.5 delivers a respective estimation for the characteristic heartbeat time of a heartbeat. Each functional unit 24.1 through 24.5 delivers a respective quality indicator Qu(1) through Qu(5) for the estimation, which the detector 25.1 through 25.5 calculates. In one embodiment, the function block 22 selects a detection result as a function of the five quality indicators Qu(1) through Qu(5), namely the detection result with the highest quality indicator. It is also possible that the function block 22 calculates a weighted mean of the estimations of the five detectors 25.1 through 25.5. The greater the quality indicator is, the greater is the weighting factor, with which an estimation is included in the weighted mean.

Exemplary detectors for characteristic heartbeat times are described in the following publications which are each incorporated herein by reference:
EP 3 600 004 B1 (corresponding to US2020100697 A 1),
L. Kahl, U. G. Hofmann: “Removal of ECG artifacts from EMG signals with different artifact magnitudes by template subtraction,” Current Directions in Biomedical Engineering, 2019, Vol. 5, No. 1, pp. 357-360,
J. Pan, W. J. Tompkins: “A real-time QRS detection algorithm,” IEEE Trans Biomedical Engineering, Vol. 32, No. 3 (1985), pp. 230-236,
A. G. Ramakrishnan, P. Prathosh, T. V. Ananthapadmanabha: “Threshold-independent QRS detection using the dynamic position index,” IEEE Signal Process Lett, Vo. 21, No. 5 (2014), pp. 554-558, and
D. S. Benitez, P. A. Gaydecki, A. Zaidi, A. P. Fitzpatrick: “A new QRS detection algorithm based on the Hilbert transform,” in: Computers in cardiology, 2000, pp. 379-382.

Such detectors can also be applied to the present invention.

It is possible that at least one detector comprises an optimal filter (matched filter), wherein such an optimal filter looks for a defined pattern in a signal and detects each time period, in which the signal has this pattern. In the present case, this pattern is the typical course of a cardiogenic signal, wherein this typical course reaches from the P peak up to the T peak or even comprises only the QRS phase, cf. FIG. 1.

An exemplary detector delivers a signal dependent on the cardiogenic signal and on the respiratory signal, which is used for the detection. Such a signal is, e.g., the signal Gm*** from L. Kahl, U. G. Hofmann, mentioned above. Detected are the intervals, in which this signal is above a first threshold value. Each maximum signal value in such an interval is used as a characteristic heartbeat point. A quality indicator depends on the following criteria:
How far is the maximum signal value in such an interval above the first threshold value?
How far is the signal below the first threshold value outside of such an interval?
In how many intervals and/or in which periods is the signal in a range between the first threshold value and a lower second threshold value?
How high is the signal to noise ratio (SNR)?

The quality indicator Qu(l) through Qu(5) depends on the implementation of the respective used detector 25.1 through 25.5. In one embodiment, additional variables are calculated, for example
the EMG to ECG ratio, i.e., the RMS(EMG)/R-S(ECG) ratio, wherein RMS(EMG) is the effective value (root mean square) of a signal of an electromyographic sensor, and R-S(ECG) is the interval between the R peak and the S peak, cf. FIG. 1 and FIG. 7, bottom,
an indicator of the regularity of the heartbeat.

As already explained, the two functional units 14 and 15 of the function block 20 continuously update the cardiogenic reference signal segment SigA_{kar.ref} and store this in the memory 9. The two functional units 14 and 15 use for this the 2*N sum signal segments of the two sum signals Sig_{Sum}[1] and Sig_{Sum}[2] for the N heartbeat time periods of the last N heartbeats x₁, . . . , x_{N}. This is preferably repeated continuously.

The two functional units 14 and 15 also require a respective characteristic heartbeat time for these N heartbeats x₁, . . . , X_{N}. This update of the cardiogenic reference signal segment SigA_{kar.ref} does not need to take place in real time, however. Therefore, more time is available to detect a heartbeat time approximately. Therefore, the result frequently has a higher reliability. The function block 22 calculates N estimations H Zp[s](x₁), H Zp[s](x_{N}) for N characteristic heartbeat times H_Zp(x ₁), H_Zp(x_{N}). The s stands for “slow.” In order to calculate these N characteristic estimations H_Zp[s](x₁), H_Zp[s](x_{N}), the functional units 14 and 15 use the respective N last sum signal segments from the five sum signals Sigₛᵤₘ[1] through Sig_{Sum}[5]. In addition, the functional units 14 and 15 apply the five detectors 25.1 through 25.5 to the five sum signals Sig_{Sum}[1] through Sig_{Sum}[5], namely a respective detector to a sum signal. Optionally, the function block 22 additionally uses the five quality indicators Qu(1), Qu(5).

Each detector 25.1 through 25.5 delivers at least one respective estimation H_Zp[1](x), H_Zp[5](x) for the actual characteristic heartbeat time H_Zp(x) of a heartbeat x. In one embodiment, the two detectors 25.1 and 25.2, which are applied to the sum signals Sig_{Sum}[1] and Sig_{Sum}[2] from the two pairs of measuring electrodes 2.1 and 2.2, two respective estimations, namely an estimation H_Zp[1.f](x), H_Zp[2.f](x) in real time and an estimation after a longer computing time H_Zp[1.s](x), H_Zp[2.s](x) and with higher accuracy. The function block 22 combines this estimation, on the one hand, in real time to form an estimation H_Zp[f](x) and, on the other hand, with longer computer time to form an additional estimation H_Zp[s](x).

As already explained, each functional unit 24.1, . . . , 24.5 delivers a respective quality indicator Qu(1), Qu(5), cf. FIG. 8. The function block 22 uses the detection results of the detectors 24.1 through 24.5 as well as the quality indicators Qu(1) through Qu(5) in order to calculate at least one estimation H_Zp[f](x), H_Zp[s](x) for the actual characteristic heartbeat time H_Zp(x). In one preferred embodiment, the function block 22 additionally delivers a respective quality indicator for each estimation. The quality indicator for an estimation is based on the quality indicators of the detection results, which the function block 22 uses for the calculation of this estimation ItZp[f](x), H_Zp[s](x). In case, for example, the detection result with the highest quality indicator is used as the estimation, the quality indicator of the estimation is equal to the quality indicator of this detection result.

A plurality of detection results can be combined (merged) to form an estimation in different ways. For example, the function block 22 forms a weighted mean value or a weighted median, wherein the weighting factors depend on the quality indicators Qu(1), Qu(n). In another embodiment, the detection result of the detector 25.i, to which the greater quality indicator Qu(2) is assigned, is used.

In the example from FIG. 8, the function block delivers, on the one hand, in real time an estimation H_Zp[t](x) for the characteristic heartbeat time H_Zp(x) as well as a quality indicator Qu[f](x) for this estimation H_Zp[f](x). This is carried out for each characteristic heartbeat time H_Zp(x), which at least one detector 25.1 through 25.5 has detected. On the other hand, the function block 22 delivers with higher accuracy and, as a result, with longer computing time a respective estimation H_Zp[s](x₁), H_Zp[s](x_{N}) for the heartbeat time H_Zp(x₁), H_Zp(x₁) and a respective quality indicator Qu[s](x₁), Qu[s](x_{N}) for this estimation H_Zp[s](x₁), H_Zp[s](x_{N}). Each quality indicator Qu[f](x), Qu[s](x) may vary from heartbeat time to heartbeat time. The compensation function block 20 uses these quality indicators Qu[f](x), Qu[s](x) to calculate the estimation Sig_{res.est}.

It is possible that a systematic offset of a detector 25.1 is determined empirically, for example, a systematic time interval between the estimation H Zp[i](x) and the actual characteristic heartbeat time H_Zp(x). This offset may, of course, be determined empirically only if a plurality of heartbeats have elapsed, and is then compensated before the combination (fusion) of the five detection results.

The detection results H_Zp[1](x), H_Zp[5](x) or H_Zp[1.s](x), H_Zp[2.s](x), H_Zp[3](x), H_Zp[5](x) of all five detectors 25.1, . . . , 25.5 are preferably combined to determine the estimation H_Zp[s](x) later, for example, only after the QRS phase, and with longer computing time and greater accuracy. Less computing time is available to calculate the estimation H_Zp[f](x) in real time. In one embodiment, the two estimations H_Zp[1.f](x) and H_Zp[2.if](x) are combined. In another embodiment, an iterative process with a stopping criterion is applied to the detection results of the two detectors 25.1 and 25.2 or even the detection results of all five detectors 25.1, . . . , 25.5. As soon as a result with a sufficiently high quality indicator is available, this result is used as the estimation H_Zp[f](x). In case a predefined time limit has elapsed, the last result of the detector obtained with the highest quality indicator or the last obtained result of a fusion is used as the estimation H_Zp[f](x). The time limit depends here on the predefined real-time requirement.

Different methods for combining (fusing) detection results are described in the following reference, which is incorporated herein by reference: C. A. Ledezma, M. Altuve: “Optimal data fusion for the improvement of QRS detection in multi-channel ECG recordings,” Medical & Biological Engineering & Computing, Vol. 57 (2019), pp. 1673-1681. These methods can in some cases also be applied as embodiments of the present invention.

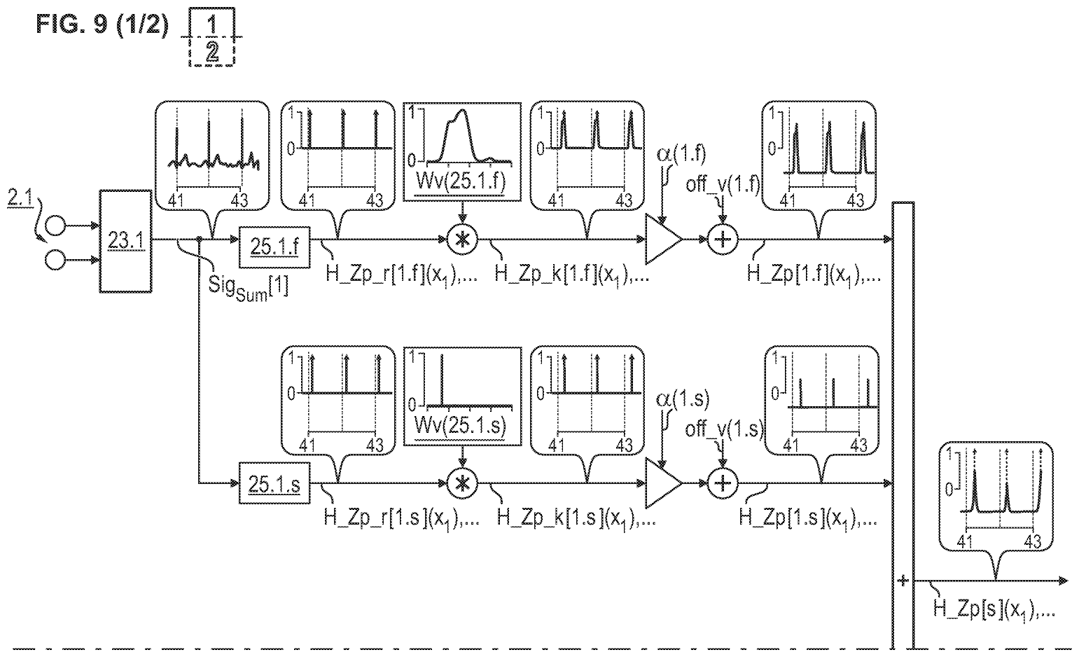

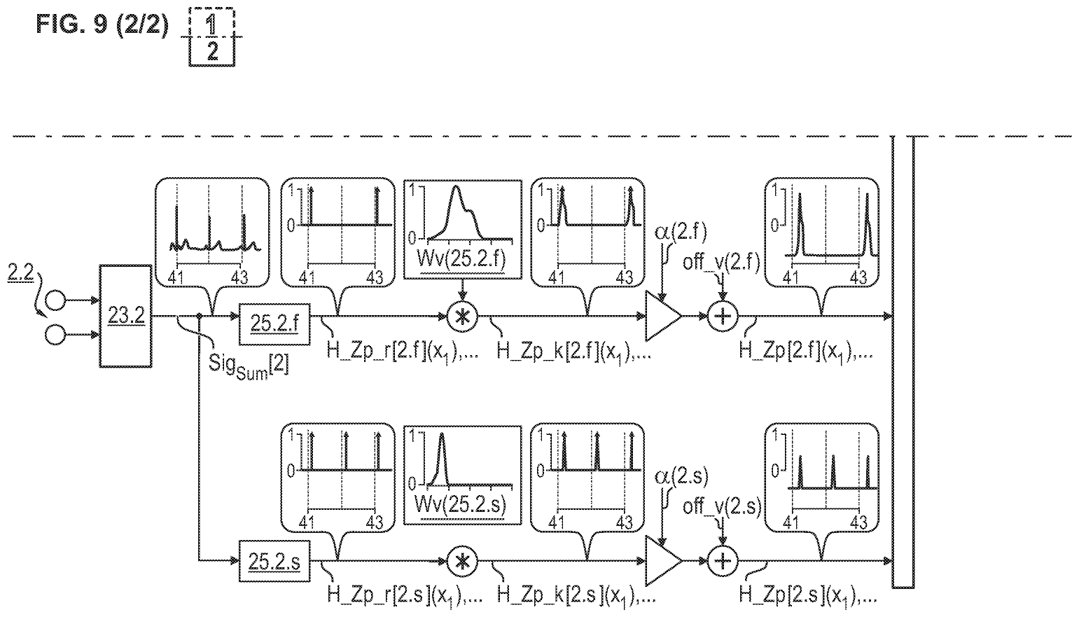

FIG. 9 illustrates, for example, an embodiment of the present invention. The two sum signals Sig_{Sum}[1] and Sig_{Sum}[2], which are obtained by processing measured values of the two pairs of measuring electrodes 2.1 and 2.2, are used in this example. These two signals are shown as examples. The two detectors 25.1.f and 25.1s deliver a respective estimation H_Zp[1.f](x₁), H_Zp[1.f](x2), . . . in real time and an estimation H_Zp[l.s](x₁), H_Zp[1.s](x₂), . . . with longer computer time, especially both times for the heartbeats x₁, x₂, . . . and based on the same sum signal Sig_{Sum}[1]. The two detectors 25.2.f and 25.2.s deliver an estimation H_Zp[2.f](x₁), H_Zp[2.f](x₂), . . . in real time and an estimation H_Zp[2.s](x₁), H_Zp[2.s](x₂), . . . with longer computing time, especially both times for the heartbeats x₁, x₂, . . . and based on the same sum signal Sig_{Sum}[2]. The two detectors 25.1.f and 25.2.f are designated as real time detectors below, the two detectors 25.1.s and 25.2.s are designated as more accurate detectors.

On the one hand, an estimation H_Zp[f](x₁), H_Zp[f](x₂), . . . is calculated and used, for example, for the control of the ventilator 1. For this, the estimations H_Zp[1.f](x₁), H_Zp[1.f](x₂), . . . , as well as H_Zp[2.f](x₁), H_Zp[2.f](x₂), . . . of the two real time detectors 25.1.f and 25.2.f are used, and optionally estimations of such detectors, which analyze other sum signals, but not the estimations H_Zp[1.s](x₁), H_Zp[1.s](x₂), . . . and H_Zp[2.s](x₁), H_Zp[2.s](x₂), . . . of the more accurate detectors 25.1.s and 25.1.s.

On the other hand, four detection results are combined, which requires, as a result, a longer computing time. This combination (fusion) is therefore used exclusively for calculating the estimation H_Zp[s](x₁), H_Zp[s](x₂), . . . How this happens is described below in an exemplary manner.

Four diagrams with three respective actual heartbeat times and the respective estimated heartbeat times are shown as examples. The time is plotted on the x axis. The characteristic heartbeat times H_Zp(x₁), H_Zp(x₂), H_Zp(x₃), which are actual and are not known in practice, are indicated by vertical broken lines. The respective sequence of the raw estimations H_Zp_r[1.f](x₁), H_Zp_r[1.f](x₂), H_Zp[2.s](x₁), H_Zp[2.s](x₂), . . . of the heartbeat times, which the four detectors 25.1.f and 25.1.s as well as 25.2.f and 25.2.s have detected, are shown by vertical solid lines with constant height, for example, the height 1 (Dirac pulse). Note: The detector 25.2.f operating in real time has not detected the second heartbeat time H_Zp(x₂).

The respective raw estimation H_Zp_r[1.f](x), H_Zp[2.s](x) for the characteristic heartbeat time deviates from the actual characteristic heartbeat time H_Zp(x). For a number of N retrograde heartbeats, it is automatically determined how great this deviation between the raw estimation and the actual time is (sign and value of the deviation). Instead of the actual heartbeat time, the respective estimation for the characteristic heartbeat time is used, which was obtained by fusion of the different estimations. An estimation for the probability distribution of the deviation is derived with a statistical method, for example, with a histogram. Four such estimated probability distributions (more precisely: derived convolution kernels) Wv(25.1f), Wv(25.2.s) for the four detectors are suggested in FIG. 9. It can be seen that the detectors 25.1.s and 25.2.s, which are operating with longer computing time, have a more narrow estimation for the probability, i.e., less scattering than the two detectors 25.1.f and 25.2.f operating in real time. These estimations are preferably updated continuously for the four probability distributions, namely on the basis of the last respective N heartbeats.

The estimated probability distribution Wv(25.1.f), . . . Wv(25.2.s) for a detector 25.1.f, . . . , 25.s.2 is combined with the sequence of estimations H_Zp[1.f](x₁), H_Zp[1.f](x₂), H_Zp[2.s](x₁), H_Zp[2.s](x₂), . . . for the heartbeat times, which this detector delivers. For example, a convolution is applied for the combination. Due to the combination, the effect of a systematic error of a detector 25.1.f, . . . , 25.s.2 is compensated by calculation. The estimation for a heartbeat time is then usually no longer a precise time, but rather is reproduced by a kind of distribution. The compensation delivers a respective compensated heartbeat time H_Zp_k[1.f](x₁), H_Zp_k[1.f](x₁), H_Zp_k[1.f](x₂), H_Zp_k[2.s](x₁), H_Zp_k[2.s](x₂),

The four signals H_Zp_k[1.f](x₁), H_Zp_k[1.f](x₂), H_Zp_k[2.s](x₁), H_Zp_k[2.s](x2), . . . of the compensated heartbeat times are subsequently attenuated with a weighting factor α(1.f), α(1.s), α(2f), α(2.s). Preferably, α(1.f)+α(1.s)+α(2.f)+α(2.s)=1. In addition, a vertical offset off_v(1.f), off_v(1.s), off_v(2.f), off_v(2.s), i.e., a constant increase or reduction of the respective signal value, is applied. This vertical offset off_v(1.f), off_v(1.s), off_v(2.f), off_v(2.s) takes into consideration the fact that the four detectors 25.1.f, 25.1.s, 25.2.f, 25.2.s deliver detection results of different quality and can be tared differently between specificity (detect no incorrect time) and sensitivity (detect each correct time).

Four signals H_Zp[1.f](x₁), H_Zp[1.f](x₂), . . . , H_Zp[2.s](x₁), H_Zp[2.s](x₂) are calculated by applying these four weighting factors a(1.0, a(1 .$), α(2.f), α(2.s) and these four offsets off_v(1.f), off_v(1.s), off_v(2.f), off_v(2.s). These four signals are added. In this signal, which is formed by addition, each maximum value is sought, which is above a predefined limit. The time corresponding to this signal value is then a heartbeat time H_Zp(x₁), H_Zp(x₂),

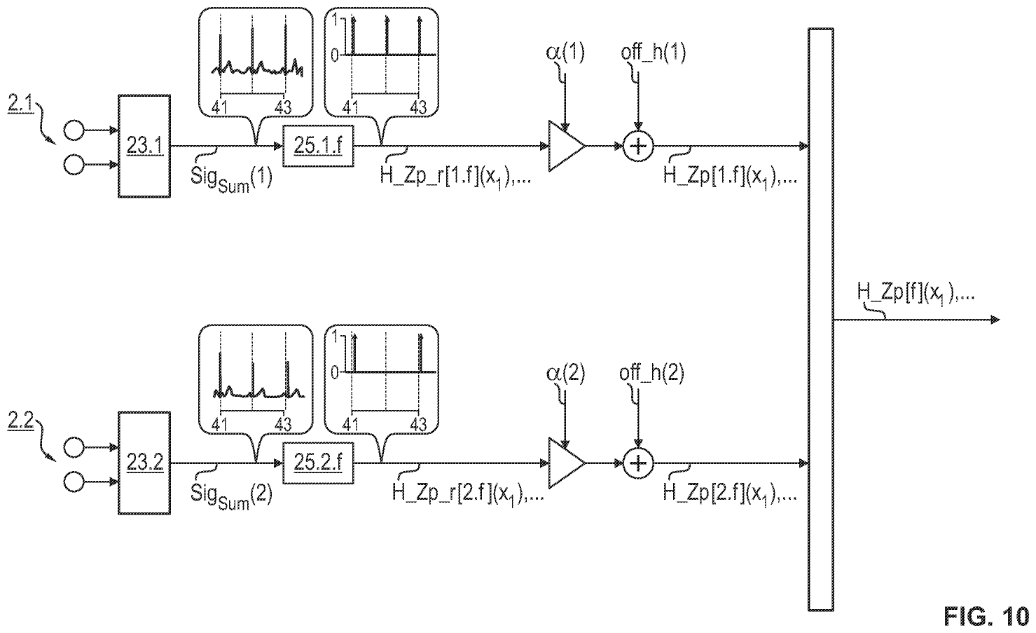

FIG. 10 shows how a respective heartbeat time H_Zp[f](x₁), H_Zp[f](x₂), . . . is detected by means of detection results H_Zp[1.f](x₁), H_Zp[1.f](x₂), H_Zp[1.s](x₁), H_Zp[1.s](x₂), of the two real time detectors 25.1.f and 25.2.f in an exemplary manner. Identical reference numbers have the same meanings as in FIG. 7. The two offsets off_h(1) and off_h(2) are horizonal offsets, i.e., time shifts, and are calculated as a function of the two estimated probability distributions Wv(25.1.f), . . . , Wv(25.2.f). A respective systematic error of a real time detector 25.1, 25.2 is compensated by calculation in this manner. The estimated probability distributions Wv(25.1.f), . . . , Wv(25.2.f) are calculated as was described with reference to FIG. 9.

While specific embodiments of the invention have been shown and described in detail to illustrate the application of the principles of the invention, it will be understood that the invention may be embodied otherwise without departing from such principles.

List of Reference Characters

| | |
|---|---|
| 1 | Ventilator; it mechanically ventilates (ventilates) and monitors the patient P; it |
| | comprises the signal processing unit 5 |
| 2.1 | Intercostal (located near the heart) pair of measuring electrodes; it comprises |
| | the measuring electrodes 2.1.1 and 2.1.2; it delivers measured values for the |
| | electrical sum signal Sig_{Sum}[1] |
| 2.1.1, 2.1.2 | Measuring electrodes of the intercostal pair 2.1 |
| 2.1 | Pair of measuring electrodes located near the diaphragm; it comprises the |
| | measuring electrodes 2.2.1 and 2.2.2; it delivers measured values for the |
| | electrical sum signal Sig_{Sum}[2] |
| 2.2.1, 2.2.2 | Measuring electrodes of the pair located near the diaphragm 2.2 |
| 3 | Pneumatic sensor in front of the mouth of the patient; it measures the volume |
| | flow Vol' and the airway pressure P_{aw} |
| 4 | Optical sensor with an image recording device and with an image processing |
| | unit; it measures the geometry of the body of the patient P, from which the |
| | current lung filling level Vol is derived by calculation |
| 5 | Signal processing unit; it comprises the function blocks 20 and 21; it carries |
| | out the steps of the process according to the present invention; it has reading |
| | access and writing access to the memory 9 |
| 6 | Probe in the trachea Sp; it measures the pneumatic pressure Pₑₛ in the trachea Sp |
| 7 | Cuff around a wrist of the patient P; it holds the catheter 17, which invasively |
| | measures the time course of the blood pressure |
| 8.1 | Sensor in the form of a finger clip on one finger of the patient P; it measures |
| | the degree of saturation of the blood with oxygen non-invasively |
| 8.2 | Sensor in the form of a finger clip on another finger of the patient P; it non- |
| | invasively measures the blood pressure of the patient P |
| 9 | Memory, to which the signal processing unit 5 has reading access and writing |
| | access and in which the cardiogenic reference signal segment SigA_{kar, ref} is |
| | stored |
| 10 | Functional unit of the compensation function block 20; it generates the |
| | synthetic cardiogenic signal Sig_{kar, syn} |
| 11 | Functional unit of the compensation function block 20; by using the synthetic |
| | cardiogenic signal Sig_{kar, syn}, it compensates the effect of the cardiogenic signal |
| | Sigₖₐᵣ on the sum signal Sig_{Sum}, for example, by subtraction of Sig_{kar, syn} |
| 12 | Functional unit of the signal processing unit 5; it detects the respective QRS |
| | interval of each heartbeat in the sum signal Sig_{Sum} |
| 13 | Functional unit of the signal processing unit 5; it detects the exact heartbeat |
| | time H_Zp(n) of each heartbeat |
| 14 | Functional unit of the compensation function block 20; it superimposes the |
| | sum signal segments for a respective heartbeat by calculation |
| 15 | Functional unit of the compensation function block 20; it generates a |
| | cardiogenic reference signal segment SigA_{kar, ref} |
| 16 | Functional unit of the compensation function block 20; it positions the |
| | cardiogenic reference signal segments SigA_{kar, ref} in a correctly timed manner |
| | as a function of the heartbeat time H_Zp(x); it combines the positioned |
| | cardiogenic reference signal segments SigA_{kar, ref} to form the synthetic |
| | cardiogenic signal Sig_{kar, syn} |
| 17 | Catheter, held by the cuff 7; it measures the time course of the blood pressure |
| | of the patient P invasively |
| 18 | Control device, which actuates the ventilator 1 and generates for this control |
| | signals comm as a function of estimated respiratory signals |
| 19 | Measured value processor; it generates the sum signal Sig_{Sum} from reinforced |
| | measured values of the sensors 2.1.1 through 2.2.2 |
| 20 | Compensation function block; it generates the synthetic cardiogenic signal |
| | Sig_{kar, syn} and the compensation signal Sig_{com} |
| 21 | Attenuation function block; it generates the estimated respiratory signal |
| | Sig_{res, est} by attenuation from the compensation signal Sig_{com} |
| 22 | Function block, which determines the respective characteristic heartbeat time |
| | H_Zp(x) of each heartbeat; it comprises the functional units 12 and 13 |
| 23.1 | Measured value processor for the intercostal pair of measuring electrodes 2.1; |
| | it sends the first sum signal Sig_{Sum}[1] |
| 23.2 | Measured value processor for the pair of measuring electrodes 2.2 located |
| | near the diaphragm; it sends the second sum signal Sig_{Sum}(2) |
| 23.3 | Measured value processor for the cuff 7; it sends the third sum signal Sig_{Sum}[3] |
| 23.4 | Measured value processor for the first sensor 8.1 for the oxygen saturation; it |
| | sends the fourth sum signal Sig_{Sum}[4] |
| 23.5 | Measured value processor for the second sensor 8.2 for the oxygen saturation; |
| | it sends the fifth sum signal Sig_{Sum}[5] |
| 24.1 | Functional unit, which calculates the quality indicator Qu(1) |
| 25.1 | Detector; it detects an estimation H_Zp[1](x) for the heartbeat time H_Zp(x) |
| | by analysis of the first sum signal Sig_{Sum}[1] |
| 25.1.f | Detector; it detects an estimation H_Zp[1.f](x) for the heartbeat time H_Zp(x) |
| | by analysis of the first sum signal Sig_{Sum}[1] in real time |
| 25.1.s | Detector; it detects an estimation H_Zp[1.s](x) for the heartbeat time H_Zp(x) |
| | by analysis of the first sum signal Sig_{Sum}[1] with longer computing time |
| comm | Control signals for actuating the ventilator 1; they are generated by the control |
| | device 18 |
| H_Zp(x) | Characteristic heartbeat time of the heartbeat x, detected approximately by the |
| | function block 22 |
| H_Zp[1](x) | Estimation detected by the detector 25.1 for the characteristic heartbeat time |
| | H_Zp(x), detected by analysis of the first sum signal Sig_{Sum}[1] |
| H_Zp[1.f](x) | Estimation detected in real time by the detector 25.1.f for the characteristic |
| | heartbeat time H_Zp(x), detected by analysis of the first sum signal Sig_{Sum}[1] |
| H_Zp_k[1.f](x) | Improvement of the estimation H_Zp_r[1.f](x) for the characteristic heartbeat |
| | time H_Zp(x), which [improvement] was generated by compensation of the |
| | systematic error of the detector 25.1.f by calculation |
| H_Zp_r[1.f](x) | Raw estimation of the detector 25.1.f for the characteristic heartbeat time H_Zp(x) |
| H_Zp[1.s](x) | Estimation detected with longer computing time by the detector 25.1.s for the characteristic |
| | heartbeat time H_Zp(x), detected by analysis of the first sum signal Sig_{Sum}[1] |
| H_Zp[f](x) | Estimation calculated in real time for the characteristic heartbeat time |
| | H_Zp(x) of the heartbeat x, calculated by the function block 22 |
| H_Zp[s](x) | Estimation calculated with higher accuracy and longer computing time for the characteristic |
| | heartbeat time H_Zp(x) of the heartbeat x, calculated by the function block 22 |
| H_Zp[2.f](x) | Estimation detected in real time by the detector 25.2.f for the characteristic |
| | heartbeat time H_Zp(x), detected by analysis of the second sum signal Sig_{Sum}[2] |
| H_Zp[2.s](x) | Estimation detected with longer computing time by the detector 25.2.s for the characteristic |
| | heartbeat time H_Zp(x), detected by analysis of the second sum signal Sig_{Sum}[2] |
| H_Zp_{ref} | Reference heartbeat time |
| H_Zr(x) | Heartbeat time period of the heartbeat x, detected by the functional unit 12 |
| H_Zr_{ref} | Reference heartbeat time period; overlapped by the cardiogenic reference |
| | signal segment SigA_{kar, ref} |
| Ip | Initialization phase; it comprises N consecutive heartbeat time periods |
| | H_Zr(x1), . . . , H_Zr(xN) |
| N | Number of heartbeat time periods of the initialization phase Ip |
| Np | Use phase, in which the cardiogenic reference signal segment SigA_{kar, ref} and |
| | the detected heartbeat times H_Zp(y1), . . . are used to generate the |
| | compensation signal Sig_{com} |
| Qu(1) | Quality indicator, which indicates with what quality the detector 24.1 detects |
| | an actual heartbeat time in the first sum signal Sig_{Sum}[1] |
| Qu(2) | Quality indicator, which indicates with what quality the detector 24.2 detects |
| | an actual heartbeat time in the first sum signal Sig_{Sum}[2] |
| Qu(3) | Quality indicator, which indicates with what quality the detector 24.3 detects |
| | an actual heartbeat time in the first sum signal Sig_{Sum}[3] |
| Qu(4) | Quality indicator, which indicates with what quality the detector 24.4 detects |
| | an actual heartbeat time in the first sum signal Sig_{Sum}[4] |
| Qu(5) | Quality indicator, which indicates with what quality the detector 24.5 detects |
| | an actual heartbeat time in the first sum signal Sig_{Sum}[5] |
| Qu[f](x) | Quality indicator for the real time estimation H_Zp[f](x) for the heartbeat time H_Zp(x) |
| Qu[s](x) | Quality indicator for the further estimation H_Zp[s](x) for the heartbeat time H_Zp(x) |
| Sig_{com} | Compensation signal; it is generated by the compensation function block 20 |
| | by compensation of the contribution of the synthetic cardiogenic signal |
| | Sig_{kar, ayn} to the sum signal Sig_{Sum} |
| Sig_{com}(x) | Signal segment of the compensation signal Sig_{com} for the heartbeat x |
| Sigₖₐᵣ | Cardiogenic signal; it brings about the cardiac activity of the patient P, it is |
| | estimated by the synthetic cardiogenic signal Sig_{kar, syn} |
| SigA_{kar, ref} | Cardiogenic reference signal segment; it describes approximately the course |
| | of the cardiogenic signal Sigₖₐᵣ during a single heartbeat; it refers to the |
| | reference heartbeat time period H_Zr_{ref} |
| SigA_{kar, ref}[1] | Cardiogenic reference signal segment, which was obtained from the sum signal Sig_{Sum}[1] |
| SigA_{kar, ref}[2] | Cardiogenic reference signal segment, which was obtained from the sum signal Sig_{Sum}[2] |
| SigA_{kar, syn}(x) | Synthetic cardiogenic signal segment for the heartbeat x, generated from the |
| | cardiogenic reference signal segment SigA_{kar, ref} by using a value of an |
| | anthropological parameter, which [value] was measured during the heartbeat x |
| Sig_{kar, syn} | Synthetic cardiogenic signal; it is an estimation for the cardiogenic signal Sigₖₐᵣ; |
| | it is generated by the functional unit 10 from the signal segments SigA_{kar, syn}(x) |
| SigA_{kar, syn}(x) | Segment of the synthetic cardiogenic signal _{Sigkar, syn} for the heartbeat x |
| Sig_{raw} | Raw signal from the sensors 2.1.1 through 2.2.2 |
| Sigᵣₑₛ | Respiratory signal to be determined; it correlates with the intrinsic breathing |
| | activity of the patient P, i.e., the breathing activity brought about by the muscles of the diaphragm |
| Sig_{res, est} | Estimation for the respiratory signal Sigᵣₑₛ to be determined |
| Sig_{res, est}[1] | Estimation for the respiratory signal Sigᵣₑₛ to be determined on the basis of the |
| | sum signal, which originates from the intercostal pair of electrodes 2.1 |
| Sig_{res, est}[2] | Estimation for the respiratory signal Sig_{rcs} to be determined on the basis of the |
| | sum signal, which originates from the pair of electrodes 2.2 located near the diaphragm |
| Sig_{Sum} | Sum signal, measured by the sum signal sensors 2.1, 2.2, 3 or 4; it results from |
| | a superimposition of the respiratory signal Sigᵣₑₛ and of the cardiogenic signal Sigₖₐᵣ |
| SigA_{Sum}(x) | Sum signal segment for the heartbeat x, generated from the sum signal Sig_{Sum}; |
| | it refers to the reference heartbeat time period H_Zr_{ref} |
| Sig_{Sum}[1] | Sum signal, which was generated from measured values of the intercostal pair 2.1 |
| Sig_{Sum}[2] | Sum signal, which was generated from measured values of the pair 2.2 located near the diaphragm |
| Sig_{Sum}[3] | Sum signal, which was generated from measured values of the cuff 7 for the blood pressure |
| Sig_{Sum}[4] | Sum signal, which was generated from measured values for the first sensor 8.1 for the oxygen saturation |
| Sig_{Sum}[5] | Sum signal, which was generated from measured values for the second sensor 8.2 for the oxygen saturation |
| Sp | Trachea of the patient P |
| Wv(25.1.f) | Estimated probability distributions (convolution kernels) for the estimations of the heartbeat time, |
| | which the detector 25.1.f delivers |

What is claimed is:

1. A process for representing a respective characteristic heartbeat time per heartbeat for a sequence of heartbeats of a patient, the process comprising the steps of:
providing a signal processing unit comprising a first detector and a second detector and a sensor arrangement comprising at least one sensor array configured to measure a variable, which correlates with cardiac activity of the patient and/or with intrinsic breathing activity of the patient;
generating at least one sum signal using measured values of the sensor array, wherein the sum signal generated or every generated sum signal comprises a superimposition of a cardiogenic signal and a respiratory signal, wherein the cardiogenic signal correlates with the cardiac activity of the patient and the respiratory signal correlates with an the intrinsic breathing activity of the patient;
calculating, with the first detector, a first detection result for the characteristic heartbeat time by analyzing the or one sum signal;
calculating, with the second detector, a second detection result for the characteristic heartbeat time by at least one of: analyzing another sum signal that is different from the sum signal analyzed by the first detector; analyzing the sum signal that is analyzed by the first detector and applying a different method of analysis than that applied by the first detector; and analyzing another sum signal that is different from the sum signal analyzed by the first detector and applying a different method of analysis than that applied by the first detector; and
with the signal processing unit calculating a representation for the characteristic heartbeat time by using the first detection result and the second detection result.

2. A process in accordance with claim 1,
wherein a cardiogenic signal segment, being a predefined cardiogenic signal segment, is given or the signal processing unit determines the cardiogenic signal segment by using a sample,
wherein the cardiogenic signal segment approximately describes a temporal course of the cardiac activity of the patient in a course of a single heartbeat,
wherein the sample comprises a plurality of segments of the sum signal or of the other sum signal,
wherein each segment of the sample refers to a respective time period, in which a single heartbeat is carried out, and
wherein the signal processing unit determines the cardiogenic signal using the detected characteristic heartbeat times and the predefined or determined cardiogenic signal segment.

3. A process in accordance with claim 1,
wherein the sensor arrangement comprises a first sensor array and a second sensor array,
wherein the first sensor array comprises at least one first sensor and the second sensor array comprises at least one second sensor,
wherein at least one of:
the second sensor is arranged at position in relation to the heart that is different from a position of the of the first sensor in relation to the heart; and
the second sensor applies a different measuring method than that applied by the first sensor, and
wherein the sum signal is a first sum signal that is generated by using measured values of the first sensor array and the other sum signal is a second sum signal that is generated by using measured values of the second sensor array;
wherein the first detector calculates the first detection result for each characteristic heartbeat time by analyzing the first sum signal; and
wherein the second detector calculates the respective second detection result for each characteristic heartbeat time by analyzing the second sum signal.

4. A process in accordance with claim 3, wherein
the signal processing unit calculates a first representation and a second representation for the respiratory signal;
the signal processing unit calculates the first representation for the respiratory signal by using the first sum signal and the respective representation for each characteristic heartbeat time; and
the signal processing unit calculates the second representation for the respiratory signal by using the second sum signal and the respective representation for each characteristic heartbeat time.

5. A process in accordance with claim 1, wherein:
the first detector comprises a first real time detector and an additional first detector and the second detector comprises a second real time detector and an additional second detector;
a calculation period is predefined,
the first real time detector calculates a first real time detection result for the characteristic heartbeat time in the calculation period;
the additional first detector calculates an additional first detection result for the characteristic heartbeat time;
the second real time detector calculates a second real time detection result for the characteristic heartbeat time in the calculation period;
the additional second detector calculates an additional second detection result for the characteristic heartbeat time;
the signal processing unit calculates a respective real time representation for each characteristic heartbeat time in the calculation period by using the first real time detection result and the second real time detection result; and
the signal processing unit calculates a respective additional representation for each characteristic heartbeat time by using the first additional detection result and the second additional detection result.

6. A process in accordance with claim 5, wherein:
wherein the sum signal is a first sum signal and the other sum signal is a second sum signal;
the first sum signal and the second sum signal are generated by using measured values of the sensor arrangement;
the second sum signal is generated by using measured values of a different sensor and/or based on a different method for processing measured values than that used to generate the first sum signal;
the first real time detector calculates the first real time detection result by using the first sum signal; and
the second real time detector calculates the second real time detection result by using the second sum signal.

7. A process in accordance with claim 5, wherein:
the signal processing unit calculates, for N heartbeats, a respective additional representation for the characteristic heartbeat times of the N heartbeats, wherein N>1 and wherein the calculated additional representation for the characteristic heartbeat times has a higher reliability than the calculated representation for each characteristic heartbeat time;
the signal processing unit calculates a respective statistical deviation indicator for the deviation between the detection result for the first detector and for the second detector for the characteristic heartbeat time and the respective additional representation for the characteristic heartbeat time, which additional representation is calculated with higher reliability;
wherein the signal processing unit calculates the statistical deviation indicator by using the N additional representations; and
wherein for the first detector and/or for the second detector, the signal processing unit corrects, by calculation, each additional detection result provided by the first detector and the second detector based on the statistical deviation indicator calculated for the first detector and for the second detector.

8. A process in accordance with claim 5, wherein:
the signal processing unit concludes the calculation of the real time representation before the heartbeat has ended; and
the signal processing unit concludes the calculation of the additional representation after the end of the heartbeat.

9. A process in accordance with claim 1, wherein
at least one of the first detector and the second detector calculates a quality indicator comprising an indicator of a reliability that the detection result or results provided by the at least one of the first detector and the second detector coincides with the characteristic heartbeat time; and
the signal processing unit calculates the representation for a characteristic heartbeat time by using the detection result or results and the quality indicator.

10. A process in accordance with claim 9, wherein the signal processing unit additionally calculates a quality indicator for the representation for a characteristic heartbeat time as a function of the quality indicators for the detection results.

11. A process in accordance with claim 1, wherein:
the signal processing unit calculates at least one representation for the respiratory signal by using the sum signal or the other sum signal;
the step of calculating the representation for the respiratory signal comprises, with the signal processing unit, compensating by calculation an effect of the cardiogenic signal on the sum signal or on the other sum signal using the detected characteristic heartbeat times.

12. A signal processing unit for representing a respective characteristic heartbeat time per heartbeat for a sequence of heartbeats of a patient, the signal processing unit comprising:
a first detector; and
a second detector, wherein the signal processing unit is configured:
to receive measured values from a sensor arrangement comprising at least one sensor array, wherein the sensor arrangement is configured to measure a variable, which correlates with at least one of cardiac activity of the patient and intrinsic breathing activity of the patient; and
to generate at least one sum signal using received measured values.
wherein the sum signal or every sum signal comprises a respective superimposition of a cardiogenic signal and of a respiratory signal,
wherein the cardiogenic signal correlates with the cardiac activity of the patient and the respiratory signal correlates with the intrinsic breathing activity of the patient,
wherein the first detector is configured to calculate a first detection result for each characteristic heartbeat time by analyzing the sum signal,
wherein the second detector is configured to calculate a respective second detection result for the characteristic heartbeat time by at least one of: analyzing another sum signal that is different from the sum signal analyzed by the first detector; analyzing the sum signal that is analyzed by the first detector and applying a different method of analysis than that applied by the first detector; and analyzing another sum signal that is different from the sum signal analyzed by the first detector and applying a different method of analysis than that applied by the first detector,
wherein the signal processing unit is configured to calculate a respective representation for the characteristic heartbeat time, and
wherein the signal processing unit is configured to calculate the representation of the characteristic heartbeat time by using the first detection result and the second detection result.

13. A signal processing unit in accordance with claim 12, wherein:
the signal processing unit comprises a first real time detector and an additional first detector as the first detector and comprises a second real time detector and an additional second detector as the second detector;
the first real time detector is configured to calculate a respective first real time detection result for the characteristic heartbeat time in a predefined calculation period,
the second real time detector is configured to calculate a respective second real time detection result for the characteristic heartbeat time in the calculation period,
the additional first detector is configured to calculate an additional first detection result for each characteristic heartbeat time,
the additional second detector is configured to calculate an additional second detection result for each characteristic heartbeat time,
the signal processing unit is configured:
to calculate a respective real time representation for the characteristic heartbeat time of a heartbeat in the calculation period by using the first real time detection result and the second real time detection result; and
to calculate a respective additional representation for the characteristic heartbeat time by using the first additional detection result and the second additional detection result.

14. A signal processing unit according to claim 12, in combination with a sensor arrangement comprising at least one sensor array configured:
to measure at least one variable, which correlates with the cardiac activity and/or with the patient's own breathing activity, wherein the signal processing unit is configured to receive measured values from the sensor array or each sensor array; and
to generate the sum signal or each sum signal by using received measured values.

15. A system comprising:
a ventilator; and
an arrangement comprising:
a signal processing unit for representing a respective characteristic heartbeat time per heartbeat for a sequence of heartbeats of a patient, the signal processing unit comprising:
a first detector; and
a second detector, wherein the signal processing unit is configured:
to receive measured values from a sensor arrangement comprising at least one sensor array, wherein the sensor arrangement is configured to measure at least one variable, which correlates with at least one of cardiac activity of the patient and intrinsic breathing activity of the patient; and
to generate at least one sum signal using received measured values,
wherein the sum signal or every sum signal comprises a respective superimposition of a cardiogenic signal and of a respiratory signal,
wherein the cardiogenic signal correlates with cardiac activity of the patient and the respiratory signal correlates with the patient's own breathing activity,
wherein the first detector is configured to calculate a first detection result for each characteristic heartbeat time by analyzing the sum signal,
wherein the second detector is configured to calculate a second detection result for each characteristic heartbeat time by at least one of: analyzing another sum signal that is different from the sum signal analyzed by the first detector; analyzing the sum signal that is analyzed by the first detector and applying a different method of analysis than that applied by the first detector; and analyzing another sum signal that is different from the sum signal analyzed by the first detector and applying a different method of analysis than that applied by the first detector,
wherein the signal processing unit is configured to calculate at least one respective representation for each characteristic heartbeat time, and
wherein the signal processing unit is configured to calculate the representation or each representation of a characteristic heartbeat time by using at least one respective first detection result and at least one respective second detection result; and
the sensor arrangement,
wherein the signal processing unit is configured to calculate a representation for the respiratory signal by using the sum signal or the other sum signal,
wherein the signal processing unit is configured to compensate an effect of the cardiogenic signal on the used sum signal or the other sum signal by using the detected characteristic heartbeat times, and
wherein the ventilator is configured to ventilate a patient including carry out ventilation as a function of the representation for the respiratory signal.
